(19) United States
(12) Patent Application Publication
(10) Pub. No.: US 2022/0249625 A1
(43) Pub. Date: Aug. 11, 2022

(54) COMPOSITIONS COMPRISING AN ENDONUCLEASE AND METHODS FOR PURIFYING AN ENDONUCLEASE

(71) Applicant: Flagship Pioneering Innovations V, Inc., Cambridge, MA (US)

(72) Inventors: Jennifer Louise Schmitke, Belmont, MA (US); Robert John Lyng, Lexington, MA (US); Carol Chan, Boston, MA (US)

(73) Assignee: Flagship Pioneering Innovations V, Inc., Cambridge, MA (US)

(21) Appl. No.: 17/425,533

(22) PCT Filed: Jan. 29, 2020
(86) PCT No.: PCT/US20/15643

§ 371 Date: Jul. 23, 2021

Related U.S. Application Data
(60) Provisional application No. 62/798,034, filed on Jan. 29, 2019.

(51) Int. Cl. A61K 38/46; C12N 9/22; C12N 15/11; C12N 5/078; C12N 15/62; C12N 15/79; A61K 35/17; A61K 35/36; A61K 35/407

(52) U.S. Cl. CPC A61K 38/465; C12N 9/22; C12N 15/11; C12N 5/0634; C12N 15/62; C12N 2800/80; A61K 35/17; A61K 35/36; A61K 35/407; C12N 2800/107; C12N 2310/20; C12N 15/79

(57) ABSTRACT

Provided are compositions comprising an endonuclease and methods for purifying an endonuclease. One aspect of the invention provides a composition comprising at least 100 mg of an untagged endonuclease having an A260/A280 absorbance ratio of from about X to about 0.8, wherein X is less than 0.8. Another aspect of the invention provides a composition, generated by contacting a composition comprising at least 100 mg of an untagged endonuclease having an A260/A280 absorbance ratio of from about X to about 0.8, wherein X is less than 0.8, with an endonuclease binding molecule, wherein the endonuclease and the endonuclease binding molecule form a protein effector.

### CROSS-REFERENCE TO RELATED APPLICATION

The present application is entitled to priority under 35 U.S.C. § 119(e) to U.S. Provisional Patent Application No. 62/798,034, filed Jan. 29, 2019, which is hereby incorporated by reference in its entirety herein.

### BACKGROUND OF THE INVENTION

Many diseases are caused by defective genes or by epigenetic changes that may affect the expression of certain genes.

### SUMMARY OF THE INVENTION

As described herein, the present invention relates to compositions comprising an endonuclease and methods for purifying an endonuclease.

One aspect of the invention provides a composition comprising at least 100 mg of an untagged endonuclease having an A260/A280 absorbance ratio of from about X to about 0.8, wherein X is less than 0.8.

Another aspect of the invention provides a composition, generated by contacting a composition comprising at least 100 mg of an untagged endonuclease having an A260/A280 absorbance ratio of from about X to about 0.8, wherein X is less than 0.8, with an endonuclease binding molecule, wherein the endonuclease and the endonuclease binding molecule form a protein effector.

Another aspect of the invention provides a method for generating and purifying a composition comprising a polypeptide. The method comprises: a) generating a composition comprising an untagged polypeptide; b) separating the polypeptide from nucleic acids or at least one impurity in the composition by a method comprising: i) contacting the polypeptide with a hydrophobic material comprising a hydrophobic side chain and eluting the polypeptide with a first solution or a first solution gradient; and/or ii) contacting the polypeptide with an ion exchange material comprising a glycosaminoglycan and eluting the polypeptide with a second solution or a second solution gradient; to obtain a composition comprising the untagged polypeptide.

Another aspect of the invention provides a composition comprising an untagged polypeptide that is produced by any of the methods disclosed herein.

Another aspect of the invention provides a composition comprising a protein effector that is produced by any of the methods disclosed herein.

Another aspect of the invention provides a pharmaceutical composition comprising any of the compositions disclosed herein.

Another aspect of the invention provides an engineered cell, comprising any of the protein effectors disclosed herein.

Another aspect of the invention provides a method for generating an engineered cell. The method comprises introducing any of the protein effectors disclosed herein into a cell.

Another aspect of the invention provides a method for treating a patient having a disease, a disorder or a condition. The method comprises administering to the patient an effective amount of a composition comprising any of the engineered cells disclosed herein.

In various embodiments of the above aspects or any other aspect of the invention delineated herein, the endonuclease has greater than about 80% purity. In certain embodiments, the endonuclease has greater than about 90% purity.

In certain embodiments, no greater than about 20% of the endonuclease is in the form of aggregates.

In certain embodiments, the composition comprises less than about 100 ng of host cell protein per mg of endonuclease.

In certain embodiments, the endonuclease is generated from a protein expression system.

In certain embodiments, the endonuclease is the polypeptide portion of a protein effector.

In certain embodiments, the endonuclease is Cas9 or a fusion protein thereof, or Cpf1 or a fusion protein thereof.

In certain embodiments, the Cas9 is a high-fidelity Cas9 (e.g. eSpCas9, SpCas9-HF1).

In certain embodiments, the Cas9 is an enzymatically inactive Cas9.

In certain embodiments, the polypeptide is a fusion polypeptide comprising Cas9 and another polypeptide. In certain embodiments, the Cas9 of the fusion polypeptide is enzymatically inactive. In certain embodiments, the Cas9 of the fusion polypeptide is enzymatically active. In certain embodiments, the another polypeptide of the fusion polypeptide is an epigenetic-modifying agent, an exonuclease or a transcriptional modulator. In certain embodiments, the epigenetic-modifying agent is a DNA methylase, a histone methyltransferase, a histone acetyltransferase, a histone deacetylase, and combinations thereof.

In certain embodiments, the Cas9 amino acid sequence is selected from the group consisting of SEQ ID NO: 1 to SEQ ID NO: 3.

In certain embodiments, the Cpf1 amino acid sequence is selected from the group consisting of SEQ ID NO: 4 to SEQ ID NO: 5.

In certain embodiments, the endonuclease activity of the composition when complexed with an endonuclease binding molecule at a 1:1 ratio is greater than about 20%.

In certain embodiments, the endonuclease binding molecule is a deoxyribonucleotide, a ribonucleotide, or a non-naturally occurring nucleotide. In certain embodiments, the endonuclease binding molecule is a guide RNA.

In certain embodiments, the endonuclease activity of the composition is greater than about 20%.

In certain embodiments, the A260/A280 absorbance ratio of the untagged polypeptide is from about X to about 0.8, wherein X is less than 0.8.

In certain embodiments, the endonuclease is an enzymatically active endonuclease or an enzymatically inactive endonuclease.

In certain embodiments, the method further comprises sonicating the composition comprising the untagged polypeptide in a lysis buffer. In certain embodiments, the lysis buffer comprises a sulfate salt, for example, ammonium sulfate or sodium sulfate. In certain embodiments, the sonication occurs after step a).

In certain embodiments, the contacting in step i) of the method occurs under conditions effective to permit binding of the polypeptide to the hydrophobic material comprising a hydrophobic side chain. In certain embodiments, the hydrophobic side chain is a octyl, phenyl, butyl, aromatic, or aliphatic side chain. In certain embodiments, the hydrophobic material comprising a hydrophobic side chain is phenyl high sub. In certain embodiments, the hydrophobic material comprising a hydrophobic side chain is comprised within a chromatography column.

In certain embodiments, the contacting in step ii) of the method occurs under conditions effective to permit binding of the polypeptide with the ion exchange material. In certain embodiments, the ion exchange material is comprised within an HPLC column.

In certain embodiments, if step i) and step ii) of the method are both carried out, step i) may precede step ii) or step ii) may precede step i).

In certain embodiments, the first solution or solution gradient comprises a salt, for example a sulfate salt, for example ammonium sulfate or sodium sulfate.

In certain embodiments, eluting the polypeptide with a first solution or solution gradient in step i) of the method comprises varying the conductivity of the solution or solution gradient.

In certain embodiments, eluting the polypeptide with a second solution or solution gradient in step ii) of the method comprises varying the conductivity of the solution or solution gradient.

In certain embodiments, the second solution or solution gradient comprises a salt, for example a sulfate salt, for example ammonium sulfate or sodium sulfate.

In certain embodiments, the method further comprises washing the untagged polypeptide on the hydrophobic material comprising a hydrophobic side chain before eluting it.

In certain embodiments, the method further comprises washing the untagged polypeptide on the ion exchange material before eluting it.

In certain embodiments, the ion exchange material comprising a glycosaminoglycan is anionic. In certain embodiments, the ion exchange material comprising a glycosaminoglycan further comprises an agarose, a sepharose or a sephadex. In certain embodiments, the glycosaminoglycan is heparin, chondroitin sulfate, dermatan sulfate, keratan sulfate, or hyaluronan.

In certain embodiments of the method, the second solution or solution gradient has a pH of from about 3.5 to about 10.

In certain embodiments, the method further comprises subjecting the untagged polypeptide eluted in step i) or ii) to purification using a hydroxyapatite resin and/or tangential flow filtration. In certain embodiments, the tangential flow filtration is conducted with a filter having a pore size of 3 kDa to 100 kDa.

In certain embodiments, the purification using a hydroxyapatite resin comprises the steps of: contacting the polypeptide with a hydroxyapatite resin material and eluting the polypeptide with a third solution or a third solution gradient; to obtain a composition comprising the untagged polypeptide.

In certain embodiments, the polypeptide is generated from a protein expression system. In certain embodiments, the protein expression system comprises a cell. In certain embodiments, the protein expression system is cell-free.

In certain embodiments, the untagged polypeptide is encoded by a vector that does not encode the polypeptide linked to a tag.

In certain embodiments, the polypeptide is the polypeptide portion of a protein effector. In certain embodiments, the polypeptide is Cas9 or a fusion protein thereof, or Cpf1 or a fusion protein thereof. In certain embodiments, the Cas9 is a high-fidelity Cas9 (e.g. eSpCas9, SpCas9-HF1). In certain embodiments, the Cas9 is an enzymatically inactive Cas9. In certain embodiments, the polypeptide is a fusion polypeptide comprising enzymatically inactive Cas9 and another polypeptide.

In certain embodiments, the method further comprises contacting the polypeptide with an endonuclease binding molecule, wherein the polypeptide and the endonuclease binding molecule form a protein effector. In certain embodiments, the endonuclease binding molecule is a deoxyribonucleotide, a ribonucleotide, or a non-naturally occurring nucleotide. In certain embodiments, the endonuclease binding molecule is a guide RNA.

In certain embodiments, the engineered cell is an immune cell or precursor cell thereof, a hepatocyte, an islet cell, or a CD34+ cell.

In certain embodiments of the method, the protein effector is introduced into the cell by electroporation, transfection, microinjection, liposome, or a vesicle.

In certain embodiments, the composition further comprises a pharmaceutically acceptable carrier or adjuvant.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 illustrates purification of NLS-Cas9-NLS over a Phenyl High Substitution column.

FIG. 2 illustrates purification of NLS-Cas9-NLS over a Heparin High Performance column.

FIG. 3 illustrates purification of NLS-Cas9-NLS over a CHT™ Hydroxyapatite column.

FIG. 4 shows SDS-PAGE results illustrating the production and purification of Cas9 using the methods disclosed herein. Aldevron® Cas9 was used as a control for comparison. CTRL: Control; WC: Whole Cell; SF: Soluble Fraction; HIC: Hydrophobic Interaction Chromatography (Phenyl High Substitution) column; Heparin: Heparin High Performance column; CHT: CHT™ Hydroxyapatite column.

### DETAILED DESCRIPTION

The present invention includes compositions and methods for purifying an endonuclease.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The term “Cas protein” refers to a CRISPR-associated protein. In some embodiments, the Cas protein can interact with a gRNA (or crRNA) and, in concert with the gRNA (or crRNA) may localize (e.g., target or home) to a site which comprises a target domain. In some embodiments, the Cas protein is capable of cleaving a target nucleic acid. A Cas protein that is capable of cleaving a target nucleic acid is referred to an enzymatically active Cas protein. A Cas protein that is incapable of cleaving a target nucleic acid or devoid of nuclease activity is referred to an enzymatically inactive Cas protein.

The term “Cas9” refers to a Cas protein that comprises a domain of, derived from, or based on the Cas9 of species described herein. The term Cas9 includes naturally derived, wild-type, recombinant, synthetically derived, modified, enzymatically active, enzymatically inactive, functional fragment thereof, or any other forms of Cas9.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides can be used for targeting cleaved double-stranded DNA.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

The term “endonuclease” refers to a class of molecules that have one or more domains that can interact with a nucleic acid and may affect cleavage thereof by cleaving internal covalent bonds linking nucleotides. In some embodiments, an endonuclease cleaves the phosphodiester bond within a polynucleotide chain.

The term “endonuclease binding molecule” as used herein refers to a molecule or moiety that binds to an endonuclease.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “protein effector” as used herein refers to a molecule or moiety that may modulate or produce a biological activity.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous agent is transferred or introduced into the host cell. A “transfected” or “transformed” or “transduced” cell is one which has been transfected, transformed or transduced with exogenous agent. The cell includes the primary subject cell and its progeny.

A “vector” is a composition of matter which comprises a nucleic acid and which can be used to deliver the nucleic acid to the interior of a cell. Numerous vectors are known in the art including, but not limited to, linear polynucleotides, polynucleotides associated with ionic or amphiphilic compounds, plasmids, and viruses. Thus, in some embodiments, the term “vector” includes an autonomously replicating plasmid or a virus. In some embodiments, the term may be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

### I. Compositions

Provided is a composition comprising a large-scale, e.g., at least 50, 100, 150, 200, 250, 300, 350, 400, 450, 500 mg or more, of a polypeptide, e.g., endonuclease, e.g., an untagged endonuclease. In some embodiments, the polypeptide, e.g., endonuclease, e.g., untagged endonuclease, e.g., Cas protein, e.g., Cas9, has an A260/A280 absorbance ratio of from about X to about 1.5, wherein X is less than about 1.5. In some embodiments, the polypeptide, e.g., endonuclease, e.g., untagged endonuclease, e.g., Cas protein, e.g., Cas9, has an A260/A280 absorbance ratio of from about X to about 1.5, wherein X is less than about 0.8. In some embodiments, the A260/A280 absorbance ratio is from about X to about 0.8, wherein X is less than 0.8. In some embodiments, the A260/A280 absorbance ratio is less than about 2.0, 1.95, 1.9, 1.85, 1.8, 1.75, 1.7, 1.65, 1.6, 1.55, 1.5, 1.45, 1.4, 1.35, 1.3, 1.25, 1.2, 1.15, 1.1, 1.05, 1.0, 0.95, 0.9, 0.85, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, or less.

In some embodiments, the composition comprises a polypeptide that has greater than about 95%, 90%, 85%, 80%, 75%, 70%, 65%, 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1% purity of the polypeptide, e.g., endonuclease, e.g., untagged endonuclease, e.g., Cas protein, e.g., Cas9.

In some embodiments, the composition comprises no greater than about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1% of the polypeptide, e.g., untagged endonuclease, e.g., endonuclease, e.g., Cas protein, e.g., Cas9, that is in the form of aggregates. In some exemplary embodiments, the aggregate is a multimer, a dimer, a trimer, a tetramer, an oligomer or a high molecular weight species. For example, the composition may include less than 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2.5%, 2.4%, 2.3%, 2.2%, 2.1%, 2%, 1.5%, 1.4%, 1.3%, 1.2%, 1.1%, 1%, 0.5%, 0.1% of aggregates.

In some embodiments, the composition comprises less than about 100 ng of host cell protein per mg of polypeptide, e.g., endonuclease, e.g., untagged endonuclease, e.g., Cas protein, e.g., Cas9. In some embodiments, the composition comprises less than about 200, 180, 160, 140, 120, 100, 90, 80, 70, 60, 50, 40, 30, 20, 10 ng or less of host cell protein per mg of polypeptide, e.g., endonuclease, e.g., untagged endonuclease, e.g., Cas protein, e.g., Cas9.

In some embodiments, the composition is substantially free of an impurity. In various embodiments, the level of the at least one impurity is reduced by at least 30%, 40%, 50%, 60%, at least 70%, at least 80%, at least 90%, or at least 95% of the at least one impurity in the sample.

In some embodiments, the impurity is a process-related impurity or a product-related substance. In some embodiments, the process-related impurity is a host cell protein, a host cell nucleic acid, a media component, or a chromatographic material. In further embodiments, the impurity is a product-related substance, such as a charge variant, an aggregate of the polypeptide of interest, a fragment of the polypeptide of interest and a modified protein, such as a denatured protein.

In one embodiment, the at least one impurity is a host cell protein. For example, the host cell protein may be reduced by at least 0.1, at least 0.15, at least 0.25, at least 0.5, at least 0.75, at least 1.0, at least 1.25 or at least 1.5 log reduction fraction.

In one embodiment, the at least one impurity is a host cell nucleic acid. For example, the host cell nucleic acid may be reduced by at least 0.1, at least 0.15, at least 0.25, at least 0.5, at least 0.75, at least 1.0, at least 1.25 or at least 1.5 log reduction fraction.

In some embodiments, the polypeptide activity, e.g., endonuclease activity, e.g., Cas protein activity, e.g., Cas9 activity, of the composition is greater than about 20% higher than that of non-purified endonuclease, as determined by an active-site titration assay or a nuclease activity assay. In some embodiments, the polypeptide activity, e.g., endonuclease activity, e.g., Cas protein activity, e.g., Cas9 activity, of the composition is greater by about 20%, 25%, 30%, 35%, 40%, 45%, 50%, 55%, 60% or more than that of non-purified endonuclease. In some embodiments, the endonuclease activity is determined by an active-site titration assay or a nuclease activity assay when substrate and endonuclease are in equal molar ratios.

In some embodiments, the polypeptide in the composition is untagged. In such embodiments, the polypeptide lacks an affinity tag. In some embodiments, the polypeptide lacks an affinity tag such as, but not limited to, histine tag (e.g., hexahistine, His6), maltose-binding protein (MBP), small ubiquitin related modifier (SUMO), NusA, thioredoxin (TrxA), glutathione S-transferase (GST), novel tags (e.g., Fh8), or any others, such as those described by Costa et. al., in Frontiers in Microbiology, February 2014, 5(63), doi: 10.3389/fmicb.2015.00063.

### A. Endonuclease

In one aspect, the composition described herein comprises a polypeptide, e.g., an endonuclease. In some embodiments, the endonuclease, e.g., untagged endonuclease, e.g., Cas protein, e.g., Cas9, is generated from a protein expression system. In some embodiments, the endonuclease, e.g., untagged endonuclease, e.g., Cas protein, e.g., Cas9, is the polypeptide portion of a protein effector.

In some embodiments, the endonuclease of the composition is a Cas protein. In some embodiments, the endonuclease is Cas9 or a modified Cas9 (e.g., enzymatically inactive Cas9) or a fragment thereof or a fusion protein thereof. In some embodiments, the endonuclease is Cpf1 or a modified Cpf1 (e.g., enzymatically inactive Cpf1) or a fragment thereof or a fusion protein thereof.

Cas9

One aspect of the invention described herein comprises an endonuclease system to target a specific nucleic acid sequence. In some embodiments, the endonuclease may include a Cas protein (e.g., Cas9) from a CRISPR/Cas system. The Cas protein may comprise at least one domain that interacts with a guide RNA (gRNA). Additionally, the Cas protein may be directed to a target sequence by a gRNA. The gRNA interacts with the Cas protein as well as the target sequence such that, once directed to the target sequence, the Cas protein is capable of cleaving the target sequence. In certain embodiments, the Cas protein is a single-protein effector. In certain embodiments, the Cas protein is one component of a multi-protein effector. In some embodiments, the Cas protein is an RNA-guided nuclease. In some embodiments, a gRNA, a sequence-specific, non-coding RNA, provides the specificity to guide the Cas protein, e.g., for the targeted cleavage, and the Cas protein may be universal and paired with different gRNAs to cleave different target sequences.

In some embodiments, the Cas protein is Cas9. One such embodiment utilizes Cas9 to bind and/or cleave DNA. In a typical CRISPR/Cas system, the Cas protein is directed to a target nucleotide sequence (e.g., a site in the genome that is to be sequence-edited) by the guide RNAs that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas protein, such as Cas9, a CRISPR RNA (“crRNA”), and a trans-activating crRNA (“tracrRNA”) with a “guide RNA”, typically about 20-ribonucleotides in length that corresponds to a target DNA sequence.

The Cas9 of the present invention can comprise a domain of, be derived from, or be based on the Cas9 species, such as Streptococcus pyogenes (spCas9), Streptococcus thermophilus (StCas9), Stapylococcus aureus (SaCas9), Neisseria meningiditis (NmCas9), Brevibacillus laterosporus (BlatCas9), Francisella novicida (FnCas9), Listeria innocua, Lactobacillus gasseri, Wolinella succinogenes, Sutterella wadsworthensis, Gamma proteobacterium, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acaryochloris marina, or other species. In some embodiments, the Cas9 may comprise a domain of, be derived from, or be based on Streptococcus pyogenes. In some embodiments, the Cas9 may comprise a domain of, be derived from, or be based on Streptococcus thermophilus. In some embodiments, the Cas9 may comprise a domain of, be derived from, or be based on Neisseria meningitidis. In some embodiments, the Cas9 may comprise a domain of, be derived from, or be based on Staphylococcus aureus. For example, Cas9 proteins are known to exist in many Type II CRISPR systems including the following as identified in the supplementary information of Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS 10270; Streptococcus pyogenes MGAS6180; Streptococcus pyogenes MGAS315; Streptococcus pyogenes SSI-1; Streptococcus pyogenes MGAS 10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAil; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405 (US Patent Application 20160298097).

In certain embodiments, the Cas9 may comprise a domain of, be derived from, or be based on Streptococcus pyogenes serotype M1 UniProtKB No. Q99ZW2 (SEQ ID NO: 1), Streptococcus thermophiles UniprotKB No. G3ECR1 (SEQ ID NO: 2), or Staphylococcus aureus UniprotKB No. J7RUA5 (SEQ ID NO: 3).

>sp|Q99ZW2|CAS9_STRP1 CRISPR-associated
endonuclease Cas9/Csn1 OS = Streptococcus
pyogenes serotype M1 OX = 301447 GN = cas9 PE = 1
SV = 1
(SEQ ID NO: 1)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
>sp|G3ECR1|CAS9_STRTR CRISPR-associated
endonuclease Cas9 OS = Streptococcus thermophilus
OX = 1308 GN = cas9 PE = 1 SV = 2
(SEQ ID NO: 2)
MLFNKCIIISINLDFSNKEKCMTKPYSIGLDIGTNSVGWAVITDNYKVPS
KKMKVLGNTSKKYIKKNLLGVLLFDSGITAEGRRLKRTARRRYTRRRNRI
LYLQEIFSTEMATLDDAFFQRLDDSFLVPDDKRDSKYPIFGNLVEEKVYH
DEFPTIYHLRKYLADSTKKADLRLVYLALAHMIKYRGHFLIEGEFNSKNN
DIQKNFQDFLDTYNAIFESDLSLENSKQLEEIVKDKISKLEKKDRILKLF
PGEKNSGIFSEFLKLIVGNQADFRKCFNLDEKASLHFSKESYDEDLETLL
GYIGDDYSDVFLKAKKLYDAILLSGFLTVTDNETEAPLSSAMIKRYNEHK
EDLALLKEYIRNISLKTYNEVFKDDTKNGYAGYIDGKTNQEDFYVYLKNL
LAEFEGADYFLEKIDREDFLRKQRTFDNGSIPYQIHLQEMRAILDKQAKF
YPFLAKNKERIEKILTFRIPYYVGPLARGNSDFAWSIRKRNEKITPWNFE
DVIDKESSAEAFINRMTSFDLYLPEEKVLPKHSLLYETFNVYNELTKVRF
IAESMRDYQFLDSKQKKDIVRLYFKDKRKVTDKDIIEYLHAIYGYDGIEL
KGIEKQFNSSLSTYHDLLNIINDKEFLDDSSNEAIIEEIIHTLTIFEDRE
MIKQRLSKFENIFDKSVLKKLSRRHYTGWGKLSAKLINGIRDEKSGNTIL
DYLIDDGISNRNFMQLIHDDALSFKKKIQKAQIIGDEDKGNIKEVVKSLP
GSPAIKKGILQSIKIVDELVKVMGGRKPESIVVEMARENQYTNQGKSNSQ
QRLKRLEKSLKELGSKILKENIPAKLSKIDNNALQNDRLYLYYLQNGKDM
YTGDDLDIDRLSNYDIDHIIPQAFLKDNSIDNKVLVSSASNRGKSDDFPS
LEVVKKRKTFWYQLLKSKLISQRKFDNLTKAERGGLLPEDKAGFIQRQLV
ETRQITKHVARLLDEKFNNKKDENNRAVRTVKIITLKSTLVSQFRKDFEL
YKVREINDFHHAHDAYLNAVIASALLKKYPKLEPEFVYGDYPKYNSFRER
KSATEKVYFYSNIMNIFKKSISLADGRVIERPLIEVNEETGESVWNKESD
LATVRRVLSYPQVNVVKKVEEQNHGLDRGKPKGLFNANLSSKPKPNSNEN
LVGAKEYLDPKKYGGYAGISNSFAVLVKGTIEKGAKKKITNVLEFQGISI
LDRINYRKDKLNFLLEKGYKDIELIIELPKYSLFELSDGSRRMLASILST
NNKRGEIHKGNQIFLSQKFVKLLYHAKRISNTINENHRKYVENHKKEFEE
LFYYILEFNENYVGAKKNGKLLNSAFQSWQNHSIDELCSSFIGPTGSERK
GLFELTSRGSAADFEFLGVKIPRYRDYTPSSLLKDATLIHQSVTGLYETR
IDLAKLGEG
>sp|JRUA5|CAS9_STAAU CRISPR-associated
endonuclease Cas9 OS = Staphylococcus
aureus OX = 1280 GN = cas9 PE = 1 SV = 1
(SEQ ID NO: 3)
MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG

The Cas protein includes any naturally derived, wild-type, recombinant, synthetically derived, modified, enzymatically active, enzymatically inactive, functional fragment of, or any other form of Cas9. For example, the Cas9 can be modified to increase nucleic acid binding affinity and/or specificity, alter an enzymatic activity, and/or change another property of the protein, e.g., solubility.

The Cas9 can also be any high-fidelity form of Cas9, for example eSpCas9 or SpCas9-HF1 (Kleinstiver et al. (2016) Nature 529; 490-495). The Cas9 can be other variant spCas9 forms including VQR, EQR, and VRER variants (Kleinstiver et al. (2015) Nature 523; 481-485). In certain embodiments, the Cas9 is codon optimized for use in humans.

In certain embodiments, the Cas9 is a catalytically dead or enzymatically inactive Cas9 (dCas9), wherein the Cas9 has reduced or lacks endonuclease activity (Qi et al. (2013) Cell 152, 1173-1183; Mali et al. (2013) Nat. Biotechnol. 31, 833-838; Maeder et al. (2013) Nat. Methods 10, 977-979). For example, the Cas9 can be modified to lack one or more functional nuclease domains (either a RuvC-like or a HNH-like nuclease domain). The Cas9 can be modified such that one or more of the nuclease domains is deleted or mutated such that it is no longer functional (i.e., the nuclease activity is absent). This can be accomplished by, for example, introducing point mutations in the two catalytic residues (e.g., D10A and H840A) of the gene encoding Cas9. In doing so, Cas9 is unable to cleave dsDNA but retains the ability to target and bind DNA.

In certain embodiments, the Cas protein can be derived from modified Cas9, e.g., dCas9. For example, the amino acid sequence of the Cas9 can be modified to alter one or more properties (e.g., nuclease activity, affinity, stability, and so forth) of the protein. Alternatively, domains of the Cas9 not involved in RNA-guided cleavage can be eliminated from the protein such that the modified Cas9 is smaller than the wild-type Cas9.

In some embodiments in which one or more of the nuclease domains is modified or inactive, the Cas9 is able to introduce a nick into a double-stranded nucleic acid (such protein is termed a “nickase”), but not cleave the double-stranded DNA (Cas9n). The invention should be construed to include any and all variants of Cas9, such as for example catalytically inactive Cas9, or single point mutations resulting in the ability to nick either the top or bottom strand, or FokI-dCas9 (Guilinger et al. (2014) Nat Biotechnol June; 32(6): 577-582). In any of the above-described embodiments, any or all of the endonuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

Cpf1

Cpf1 is an RNA-guided nuclease that is smaller than Cas9 and is derived from the type V CRISPR system (CRISPR from Prevotella and Francisella). Like Cas9, Cpf1 family members contain a RuvC-like endonuclease domain, but they lack the second HNH endonuclease domain of Cas9. Cpf1 cleaves DNA in a staggered pattern and requires only one RNA rather than the two (tracrRNA and crRNA) needed by Cas9 for cleavage. Specifically, Cpf1 does not require a tracrRNA and can mediate target cleavage with a single crRNA. The Cpf1 enzyme can be derived from any genera of microbes, including but not limited to, Parcubacteria, Lachnospiraceae, Butyrivibrio, Peregrinibacteria, Acidaminococcus, Porphyromonas, Lachnospiraceae, Porphromonas, Prevotella, Moraxela, Smithella, Leptospira, Lachnospiraceae, Francisella, Candidatus, and Eubacterium. In certain embodiments, Cpf1 is derived from a species from the Acidaminococcus genus (AsCpf1). In other embodiments, Cpf1 is derived from a species from the Lachnospiraceae genus (LbCpf1). In yet other embodiments, the Cpf1 is a humanized form of Cpf1.

In certain embodiments, the endonuclease may comprise a domain of, be derived from, or be based on Cpf1 from Lachnospiraceae bacterium ND2006 UniprotKB No. A0A182DWE3 (SEQ ID NO: 4), or Acidaminococcus sp. (strain BV3L6) UniprotKB No. U2UMQ6 (SEQ ID NO: 5).

>tr|A0A182DWE3|A0A182DWE3_9FIRM Cpf1 OS =
Lachnospiraceae bacterium ND2006 OX = 1410628
PE = 1 SV = 1
(SEQ ID NO: 4)
AASKLEKFTNCYSLSKTLRFKAIPVGKTQENIDNKRLLVEDEKRAEDYKG
VKKLLDRYYLSFINDVLHSIKLKNLNNYISLFRKKTRTEKENKELENLEI
NLRKEIAKAFKGAAGYKSLFKKDIIETILPEAADDKDEIALVNSFNGFTT
AFTGFFDNRENMFSEEAKSTSIAFRCINENLTRYISNMDIFEKVDAIFDK
HEVQEIKEKILNSDYDVEDFFEGEFFNFVLTQEGIDVYNAIIGGFVTESG
EKIKGLNEYINLYNAKTKQALPKFKPLYKQVLSDRESLSFYGEGYTSDEE
VLEVFRNTLNKNSEIFSSIKKLEKLFKNFDEYSSAGIFVKNGPAISTISK
DIFGEWNLIRDKWNAEYDDIHLKKKAVVTEKYEDDRRKSFKKIGSFSLEQ
LQEYADADLSVVEKLKEIIIQKVDEIYKVYGSSEKLFDADFVLEKSLKKN
DAVVAIMKDLLDSVKSFENYIKAFFGEGKETNRDESFYGDFVLAYDILLK
VDHIYDAIRNYVTQKPYSKDKFKLYFQNPQFMGGWDKDKETDYRATILRY
GSKYYLAIMDKKYAKCLQKIDKDDVNGNYEKINYKLLPGPNKMLPKVFFS
KKWMAYYNPSEDIQKIYKNGTFKKGDMFNLNDCHKLIDFFKDSISRYPKW
SNAYDFNFSETEKYKDIAGFYREVEEQGYKVSFESASKKEVDKLVEEGKL
YMFQIYNKDFSDKSHGTPNLHTMYFKLLFDENNHGQIRLSGGAELFMRRA
SLKKEELVVHPANSPIANKNPDNPKKTTTLSYDVYKDKRFSEDQYELHIP
IAINKCPKNIFKINTEVRVLLKHDDNPYVIGIDRGERNLLYIVVVDGKGN
IVEQYSLNEIINNFNGIRIKTDYHSLLDKKEKERFEARQNWTSIENIKEL
KAGYISQVVHKICELVEKYDAVIALEDLNSGFKNSRVKVEKQVYQKFEKM
LIDKLNYMVDKKSNPCATGGALKGYQITNKFESFKSMSTQNGFIFYIPAW
LTSKIDPSTGFVNLLKTKYTSIADSKKFISSFDRIMYVPEEDLFEFALDY
KNFSRTDADYIKKWKLYSYGNRIRIFAAAKKNNVFAWEEVCLTSAYKELF
NKYGINYQQGDIRALLCEQSDKAFYSSFMALMSLMLQMRNSITGRTDVDF
LISPVKNSDGIFYDSRNYEAQENAILPKNADANGAYNIARKVLWAIGQFK
KAEDEKLDKVKIAISNKEWLEYAQTSVK
>sp|U2UMQ6|CS12A_ACISB CRISPR-associated
endonuclease Cas12a OS = Acidaminococcus sp.
(strain BV3L6) OX = 1111120 GN = cas12a PE = 1
SV = 1
(SEQ ID NO: 5)
MTQFEGFTNLYQVSKTLRFELIPQGKTLKHIQEQGFIEEDKARNDHYKEL
KPIIDRIYKTYADQCLQLVQLDWENLSAAIDSYRKEKTEETRNALIEEQA
TYRNAIHDYFIGRTDNLTDAINKRHAEIYKGLFKAELFNGKVLKQLGTVT
TTEHENALLRSFDKFTTYFSGFYENRKNVFSAEDISTAIPHRIVQDNFPK
FKENCHIFTRLITAVPSLREHFENVKKAIGIFVSTSIEEVFSFPFYNQLL
TQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAE
ALFNELNSIDLTHIFISHKKLETISSALCDHWDTLRNALYERRISELTGK
ITKSAKEKVQRSLKHEDINLQEIISAAGKELSEAFKQKTSEILSHAHAAL
DQPLPTTLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

The invention should be construed to include any and all forms of Cpf1 (e.g., wild type or mutant or fragments thereof). For example, the Cpf1 can be a high-fidelity Cpf1. In some embodiments, one or more domains of or fragments of Cpf1 is fused to one or more other moieties, such as those described elsewhere herein.

In another embodiment, the Cpf1 can be an enzymatically inactive form of Cpf1, e.g., DNase-dead Cpf1 (ddCpf1) (Zhang et al. (2017) Cell Discovery volume 3, Article number: 17018), or dAsCpf1 (Liu et al. (2017) Nature Communications volume 8, Article number: 2095). In some embodiments, the Cpf1 is modified to an enzymatically reduced form.

Endonuclease Fusions

In certain embodiments, the endonuclease is a fusion protein. The Cas-like portion of the fusion protein can be derived from any form of Cas9, e.g., a wild-type Cas9 or Cpf1, a domain or a fragment thereof, or a modified Cas9, e.g., dCas9.

In certain embodiments, the endonuclease is part of a fusion protein comprising one or more heterologous moieties (e.g. about or more than about 1, 2, 3, 4, 5, 6, 7, 8, 9, 10, or more moieties in addition to the endonuclease). An endonuclease fusion may comprise one or more additional moieties, and optionally a linker sequence between any two moieties. Examples of moieties that may be fused to the endonuclease include, without limitation, epitope tags, reporter gene sequences, and protein domains having one or more of the following activities: nuclear localization activity, methylase activity, demethylase activity, transcription activation activity, transcription repression activity, transcription release factor activity, histone modification activity, RNA cleavage activity and nucleic acid binding activity. Additional moieties that may form part of a fusion protein comprising the endonuclease are described in U.S. Patent Appl. Publ. No. US20110059502, incorporated herein by reference in its entirety.

In some embodiments, the endonuclease is Cas9 and Cas9 is fused to one or more moieties. In further embodiments, the one or more moieties comprises an epigenetic-modifying agent. In yet further embodiments, the epigenetic-modifying agent comprises a DNA methylase, a histone methyltransferase, a histone deacetylase, or combinations thereof.

In some embodiments, the endonuclease described herein is a fusion protein with one or more modulating or effector moieties, e.g., a moiety capable of modulating nucleic acids, including KRAB, MQ1, DMNT3A, DMNT3L, APOBECs, and FOK1.

In some embodiments, the endonuclease (e.g., molecule, fragment, or domain thereof) fusion comprises a transcriptional modulator (e.g., a molecule, a fragment, or a domain thereof). Those skilled in the art are familiar with transcriptional modulators would understand that transcriptional modulators include a variety of positive (e.g., enhancers) or negative (e.g., repressors or silencers) transcriptional modulators that are associated with gene transcription. In certain instances, binding a transcriptional modulator to a gene results in altered transcription, e.g., increased for a positive transcriptional modulator; decreased for a negative transcriptional modulator.

In certain embodiments, a protein effector causing a transcriptional modification comprises a fusion polypeptide comprising a transcriptional modifying agent. In certain embodiments, the protein effector comprises a fusion polypeptide comprising an enzymatically inactive Cas9 polypeptide and an epigenetic modifying agent, or a nucleic acid encoding the fusion polypeptide. In certain embodiments, the protein effector comprises a fusion polypeptide comprising an enzymatically inactive Cpf1 polypeptide and an epigenetic modifying agent, or a nucleic acid encoding the fusion polypeptide. In certain embodiments, the protein effector comprises a fusion polypeptide comprising an enzymatically active Cas9 polypeptide and an epigenetic modifying agent, or a nucleic acid encoding the fusion polypeptide. In further embodiments, the epigenetic modifying agent is an exonuclease, e.g. exonuclease 1 (EXO1). In certain embodiments, the protein effector comprises a fusion polypeptide comprising an enzymatically active Cpf1 polypeptide and an epigenetic modifying agent, or a nucleic acid encoding the fusion polypeptide. In further embodiments, the epigenetic modifying agent is an exonuclease, e.g. exonuclease 1 (EXO1).

In certain embodiments, the protein effector causing an epigenetic modification comprises a fusion protein and a gRNA.

In some embodiments, the protein effector causing the epigenetic modification comprises a fusion polypeptide. In some embodiments, the fusion polypeptide comprises a Cas protein (e.g., Cas9, enzymatically inactive Cas9) and a polypeptide. In some embodiments, the polypeptide is an epigenetic modifying agent. In some embodiments, the polypeptide is selected from the group consisting of epigenetic enzymes (DNA methylases (e.g., DNMT3a, DNMT3b, DNMTL), DNA demethylases (e.g., the TET family), histone methyltransferases, histone acetyltransferase (e.g., P300), VP64, VP64 transactivation domain, KRAB, histone deacetylase (e.g., HDAC1, HDAC2, HDAC3), sirtuin-1, -2, -3, -4, -5, -6, or -7, lysine-specific histone demethylase 1 (LSD 1), histonelysine-N-methyltransferase (Setdb 1), euchromatic histone-lysine N-methyltransferase 2 (G9a), histone-lysine N-methyltransferase (SUV39H 1), enhancer of zeste homolog 2 (EZH2), viral lysine methyltransferase (vSET), histone methyltransferase (SET2), and protein-lysine N-methyltransferase (SMYD2), a fusion of a sequence targeting polypeptide and a conjunction nucleating molecule, and any combination thereof.

In certain embodiments, the protein effector comprises a Cas fusion protein comprising a first polypeptide comprising a Cas or modified Cas protein domain and a second polypeptide comprising a polypeptide having DNA methyltransferase activity (or associated with demethylation or deaminase activity), and at least one guide RNA (gRNA) that targets the protein to an anchor sequence of a target anchor sequence-mediated conjunction. The phrase “anchor sequence-mediated conjunction” as used herein, refers to a DNA structure, in some cases, a loop, that occurs and/or is maintained via the physical interaction or binding of at least two anchor sequences in the DNA by one or more proteins, such as nucleating proteins, or one or more proteins and/or a nucleic acid entity (such as RNA or DNA), that bind the anchor sequences to enable spatial proximity and functional linkage between the anchor sequences.

### B. Endonuclease Binding Molecule

In some embodiments, a composition comprises an endonuclease (e.g. Cas9) and an endonuclease binding molecule. In some embodiments, the endonuclease binding molecule is a DNA-binding oligonucleotide. In some embodiments, the endonuclease binding molecule comprises a nucleotide sequence that is complementary to, and will bind with, a ‘target’ DNA sequence. In some embodiments, the endonuclease binding molecule is a guide RNA (gRNA). In some embodiments, the gRNA is inactive (e.g. dgRNA). In some embodiments, the endonuclease binding molecule is a CRISPR RNA (crRNA). In some embodiments, the endonuclease binding molecule is a crRNA and a trans-activating CRISPR RNA (tracrRNA). In some embodiments, the endonuclease binding molecule comprises a modified nucleic acid sequence. In some embodiments, the endonuclease binding molecule comprises a nucleic acid sequence with an alternate backbone. In some embodiments, the endonuclease binding molecule comprises a chemical compound or other non-nucleic acid moiety that mimics nucleic acid binding to the endonuclease, e.g., a glycosaminoglycan, e.g., chondroitin sulfate, dermatan sulfate, keratan sulfate, heparin, or hyaluronan.

In some embodiments, an endonuclease binding moiety comprises a gRNA. In some embodiments, an endonuclease binding moiety is a molecule that the endonuclease binds. In some embodiments, the endonuclease binding molecule may comprise a targeting sequence that is complementary to and hybridizes with the target sequence on the target nucleic acid molecule. The endonuclease binding molecule can be designed to target any sequence of interest. The target sequence of the endonuclease binding molecule may be within some loci of a gene or within a non-coding region of a genome. In some embodiments, the endonuclease binding molecule may parallel the structure of a gRNA or crRNA.

In some embodiments, the endonuclease binding molecule and the endonuclease may form a protein effector, e.g., a Cas complex. The endonuclease binding molecule may guide the endonuclease to a target sequence on a target nucleic acid molecule, where the endonuclease binding molecule binds to the target nucleic acid molecule and the endonuclease cleaves the target sequence.

In some embodiments, the endonuclease binding molecule is a crRNA. In some embodiments, the endonuclease is Cpf1 and the endonuclease binding molecule is crRNA. In some embodiments, the endonuclease and the endonuclease binding molecule form a protein effector.

In some embodiments, the endonuclease binding molecule is a gRNA. In some embodiments, the endonuclease is a Cas protein, and the endonuclease binding molecule is a gRNA, forming a Cas complex. In some embodiments, the Cas protein may be a Cas9 protein. In some embodiments, the Cas complex may be a Cas9/gRNA complex. The terms sgRNA and gRNA are used interchangeably herein.

In some embodiments, the endonuclease binding molecule provided herein comprises a targeting domain comprising, consisting of, or consisting essentially of a nucleic acid sequence fully or partially complementary to a target domain (also referred to as “target sequence”). The endonuclease binding molecule may be specific for a genomic region of interest and target that region for Cas protein-induced double strand breaks. In some embodiments, the endonuclease binding molecule is designed to have a length of between 17-24 nucleotides (e.g., 17, 18, 19, 20, 21, 22, 23, or 24 nucleotides) and have complementarity to a targeted gene or nucleic acid sequence. In certain embodiments, the endonuclease binding molecule is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length. Custom endonuclease binding molecule generators and algorithms are available commercially for use in the design of effective endonuclease binding molecules, e.g., gRNAs. In certain embodiments, a 21-nucleotide sequence is used as input into the CRISPR-Cpf1 crRNA ordering tool (e.g., from Integrated DNA Technologies, Inc. of Skokie, Ill. and available at www.idtdna.com/CRISPR-Cpf1). In certain embodiments, the endonuclease binding molecule is at least 40, 41, 42, 43, 44 or more nucleotides in length.

In certain embodiments, the endonuclease binding molecule can comprise a catalytically dead guide RNA (dgRNA). These dgRNAs are typically shortened in length (14-nt or 15-nt) and are catalytically inactive yet maintain target-site binding capacity (Kiani et al. (2015) Nat Methods 12, 1051-1054; Dahlman et al. (2015) Nat Biotechnol 33(11): 1159-1161). Thus, these dgRNAs can be utilized to modulate gene expression using a catalytically active Cas9.

Any of the endonuclease binding molecules described herein can be generated by in vitro transcription or chemical synthesis or any means known to one of ordinary skill in the art.

Chemically modified endonuclease binding molecules have also been demonstrated to be effective in genome editing; see, for example, Hendel et al. (2015) Nature Biotechnol., 985-991.

In some embodiments, the endonuclease binding molecule comprises one or more modifications, e.g., a modified nucleic acid. Modifications include, for example, end modifications, e.g., 5′-end modifications (phosphorylation (mono-, di- and tri-), conjugation, inverted linkages, etc.), 3′-end modifications (conjugation, DNA nucleotides, inverted linkages, etc.), base modifications (e.g., replacement with stabilizing bases, destabilizing bases, or bases that base pair with an expanded repertoire of partners), removal of bases (basic nucleotides), or conjugated bases. In some embodiments, a modified ribonucleotide base can also include a 5-methylcytidine or pseudouridine. In some embodiments, base modifications can modulate binding, stability, specificity, to name a few functional effects of the endonuclease binding molecule. In some embodiments, the modification includes a bi-orthogonal nucleotide, e.g., an unnatural base.

In some embodiments, modifications may include sugar modifications (e.g., at the 2′ position or 4′ position) or replacement of the sugar in one or more ribonucleotides, backbone modifications in one or more ribonucleotides, and modification or replacement of the phosphodiester linkages of the endonuclease binding molecule. Non-limiting examples of endonuclease binding molecules include modified backbones or non-natural internucleoside linkages, such as those modified or replaced of the phosphodiester linkages. Endonuclease binding molecules having modified backbones include, among others, those that do not have a phosphorus atom in the backbone. For the purposes of this application, and as sometimes referenced in the art, modified RNA that do not have a phosphorus atom in their internucleoside backbone can also be considered to be oligonucleosides. In particular embodiments, the endonuclease binding molecule includes ribonucleotides with a phosphorus atom in its internucleoside backbone.

Modified endonuclease binding molecule backbones can include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the endonuclease binding molecule can be negatively or positively charged.

Modified nucleotides, which can be incorporated into the endonuclease binding molecule, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

In some embodiments, an α-thio substituted phosphate moiety is incorporated to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the endonuclease binding molecule is expected to reduce the innate immune response through weaker binding/activation of cellular innate immune molecules.

In some embodiments, a modified nucleoside includes an α-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine). Other internucleoside linkages can include internucleoside linkages which do not contain a phosphorous atom.

In some embodiments, the endonuclease binding molecule can include one or more cytotoxic nucleosides. For example, cytotoxic nucleosides can be incorporated into the endonuclease binding molecule, such as bifunctional modification. Cytotoxic nucleoside can include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((R,S)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

The endonuclease binding molecule can be uniformly modified along the entire length of the moiety or in select sites of the endonuclease binding molecule. For example, one or more or all types of nucleotides (e.g., naturally-occurring nucleotides, purine or pyrimidine, or any one or more or all of A, G, U, C, I, pU) can be uniformly modified in the endonuclease binding molecule, or in a given predetermined sequence region thereof. In some embodiments, the endonuclease binding molecule includes a pseudouridine. In some embodiments, the endonuclease binding molecule includes an inosine, which can aid in the immune system characterizing the endonuclease binding molecule as endogenous versus viral RNA. The incorporation of inosine can also mediate improved stability/reduced degradation.

In some embodiments, all nucleotides in the endonuclease binding molecule (or in a given sequence region thereof) are modified. In some embodiments, the modification can include an inosine, which can attenuate an immune response; pseudouridine, which can increase RNA stability, an m5C, which can increase stability; and a 2,2,7-trimethylguanosine, which aids subcellular translocation (e.g., nuclear localization).

Different sugar modifications, nucleotide modifications, and/or internucleoside linkages (e.g., backbone structures) can exist at various positions in the endonuclease binding molecule. One of ordinary skill in the art will appreciate that the nucleotide analogs or other modification(s) can be located at any position(s) of the endonuclease binding molecule, such that function is not substantially decreased. The endonuclease binding molecule can include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, the endonuclease binding molecule is a chemical or other non-nucleic acid moiety. In some embodiments, the endonuclease binding molecule comprises a chemical compound or other non-nucleic acid moiety that mimics nucleic acid binding to the endonuclease, e.g., a glycosaminoglycan, e.g., chondroitin sulfate, dermatan sulfate, keratan sulfate, heparin, or hyaluronan. In some embodiments, the endonuclease binding molecule comprises a generically sulfated mucin or other negatively charged molecule. In some embodiments, the endonuclease binding molecule comprises a natural or synthetic chemical or other non-nucleic 8-mer moiety.

### C. A260/A280 Absorbance Ratio

The ratio of absorbance at 260 nm vs 280 nm (A260/A280 ratio) is commonly used to assess nucleic acid contamination of polypeptide solutions, since polypeptides absorb light at 280 nm, largely due to their aromatic amino acids, while nucleic acids absorb light at 260 nm. The purer a polypeptide solution is, the higher the A280 will be.

To calculate the A260/A280 ratio of a composition, the absorbance at 260 nm (A260) is divided by the absorbance at 280 nm (A280) to yield a value (v). The A260/A280 ratio is then expressed as v.

In some embodiments, the polypeptide, e.g., untagged endonuclease, e.g., Cas protein, e.g., Cas9, has an A260/A280 absorbance ratio of from about X to about 1.5, wherein X is less than about 1.5. In some embodiments, the A260/A280 absorbance ratio of the composition comprising an untagged polypeptide is from about X to about 0.8, wherein X is less than 0.8. In further embodiments, the A260/A280 absorbance ratio of the composition comprising an untagged polypeptide is from about 0.8 to about 2.0. In some embodiments, the A260/A280 absorbance ratio is less than about 2.0, 1.95, 1.9, 1.85, 1.8, 1.75, 1.7, 1.65, 1.6, 1.55, 1.5, 1.45, 1.4, 1.35, 1.3, 1.25, 1.2, 1.15, 1.1, 1.05, 1.0, 0.95, 0.9, 0.85, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, or less.

In some embodiments, A260/A280 ratio may be determined or measured by solution UV scan, or slope spectroscopy. The A260/A280 ratio may be determined by measuring the absorbance of a solution using a spectrophotometer. Spectrophotometric analysis is based on the principles that nucleic acids absorb ultraviolet light in a specific pattern. When a sample is exposed to ultraviolet light at a wavelength of 260 nm, a photo-detector measures the light that passes through the sample. Some of the light is absorbed by the DNA or RNA, and some of the light passes through the sample. The more light at a wavelength of 260 nm absorbed by the sample, the higher the nucleic acid concentration in the sample, the less light will strike the photodetector, and the higher the resulting optical density (OD) will be.

When a sample is exposed to ultraviolet light at 280 nm, some of the light is absorbed by polypeptide in the sample. The more light at a wavelenth of 280 nm absorbed by the sample, the higher the protein concentration in the sample, the less light will strike the photodetector, and the higher the resulting optical density (OD) will be.

Spectrophotometers that may be used to determine the A260/A280 ratio include but are not limited to ultraviolet-visible, near-infrared, and fluorescent spectrophotometers.

Methods of determining the A260/A280 ratio are known to a person of skill in the art. In some embodiments, the A260/A280 ratio is determined by solution UV scan or by SOLOVPE® apparatus. In further embodiments, the A260/A280 ratio is determined by SEC-MALS (size exclusion chromatography-multiangle light scattering).

### D. Composition Characteristics

In some embodiments, the composition comprises no greater than about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1% of the polypeptide, e.g., endonuclease, e.g., untagged endonuclease, e.g., Cas protein, e.g., Cas9, is denatured as determined by size exclusion chromatography multiangle static light scattering (e.g., SEC-MALS). In further embodiments, the polypeptide, e.g., endonuclease, e.g., untagged endonuclease, e.g., Cas protein, e.g., Cas9, is not denatured.

In some embodiments, the composition comprises no greater than about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1% of the polypeptide, e.g., endonuclease, e.g., untagged endonuclease, e.g., Cas protein, e.g., Cas9, is in the form of aggregates. In some embodiments, aggregate measurements may be determined by SEC-MALS, gel electrophoresis (e.g., SDS-PAGE), or HPLC. In some exemplary embodiments, the aggregate is a multimer, a dimer, a trimer, a tetramer, an oligomer or a high molecular weight species. For example, the composition may include less than 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2.5%, 2.4%, 2.3%, 2.2%, 2.1%, 2%, 1.5%, 1.4%, 1.3%, 1.2%, 1.1%, 1%, 0.5%, 0.1% of aggregates.

In some embodiments, the composition comprises less than about 100 ng of host cell protein per mg of polypeptide, e.g., endonuclease, e.g., untagged endonuclease, e.g., Cas protein, e.g., Cas9. In some embodiments, concentration of the endonuclease is measure by solution UV scan, slope spectroscopy, SEC-MALS, or HPLC. In some embodiments, the composition comprises less than about 200, 180, 160, 140, 120, 100, 90, 80, 70, 60, 50, 40, 30, 20, 10 ng or less of host cell protein per mg of polypeptide, e.g., endonuclease, e.g., untagged endonuclease, e.g., Cas protein, e.g., Cas9.

In some embodiments, the composition is substantially free of an impurity. In some embodiments, one or more impurities are measured by HPLC, quantitative ELISA, immunodetection of host proteins, or bio-layer interferometry. In various embodiments, the level of the at least one impurity is reduced by at least 30%, 40%, 50%, 60%, at least 70%, at least 80%, at least 90%, or at least 95% of the at least one impurity in the sample.

In some embodiments, the impurity is a process-related impurity or a product-related substance. In some embodiments, the process-related impurity is a host cell protein, a host cell nucleic acid, a media component, or a chromatographic material. In further embodiments, the impurity is a product-related substance, such as a charge variant, an aggregate of the polypeptide of interest, a fragment of the polypeptide of interest and a modified protein, such as a denatured protein.

In one embodiment, the at least one impurity is a host cell protein. For example, the host cell protein may be reduced by at least 0.1, at least 0.15, at least 0.25, at least 0.5, at least 0.75, at least 1.0, at least 1.25 or at least 1.5 log reduction fraction.

In one embodiment, the at least one impurity is a host cell nucleic acid. For example, the host cell nucleic acid may be reduced by at least 0.1, at least 0.15, at least 0.25, at least 0.5, at least 0.75, at least 1.0, at least 1.25 or at least 1.5 log reduction fraction.

In some embodiments, the polypeptide activity, e.g., endonuclease activity, e.g., Cas protein activity, e.g., Cas9 activity, of the composition is greater than about 20% than non-purified endonuclease, as determined by an active-site titration assay or a nuclease activity assay. In some embodiments, the polypeptide activity, e.g., endonuclease activity, e.g., Cas protein activity, e.g., Cas9 activity, of the composition is greater than about 20%, 25%, 30%, 35%, 40%, 45%, 50%, 55%, 60% or more than non-purified endonuclease. In some embodiments, the endonuclease activity is determined by an active-site titration assay or a nuclease activity assay when substrate and endonuclease are in equal molar ratios.

### II. Methods for Generating the Composition

Provided is a method for generating and purifying a composition comprising a polypeptide, the method comprising:

a) generating a composition comprising an untagged polypeptide;

b) separating the polypeptide from nucleic acids or at least one impurity in the composition by a method comprising:
i) contacting the polypeptide with a hydrophobic material comprising an aromatic side chain and eluting the polypeptide with a first solution or a first solution gradient; and/or
ii) contacting the polypeptide with an ion exchange material comprising a glycosaminoglycan and eluting the polypeptide with a second solution or a second solution gradient;
   to obtain a composition comprising the untagged polypeptide.

In some embodiments, the A260/A280 absorbance ratio of the untagged polypeptide composition is from about X to about 0.8, wherein X is less than 0.8.

In some embodiments, the purity of the composition when complexed at a 1:1 ratio with an endonuclease binding molecule is greater than about 85%, 90%, 91%, 92%, 93%, 94%, 95%, 96%, 97%, 98% or 99% as determined by SEC-MALS, SDS-PAGE or HPLC-RP. In further embodiments, the purity of the composition is greater than about 98%.

### A. Procedures and Protein Expression Systems for Generating Untagged Endonuclease

Methods of making the polypeptide described herein, e.g., endonuclease, are routine in the art. See, in general, Voynov & Caravella (Eds.), Therapeutic Proteins: Methods and Protocols (Methods in Molecular Biology) Second Edition, Humana Press (2012); and Crommelin, Sindelar & Meibohm (Eds.), Pharmaceutical Biotechnology: Fundamentals and Applications, Springer (2013). In some embodiments, the therapeutic protein or polypeptide is an endonuclease.

The polypeptide, e.g., endonuclease, can be biochemically synthesized by employing standard solid phase techniques. Such methods include, for example, exclusive solid phase synthesis, partial solid phase synthesis methods, fragment condensation, and classical solution synthesis. These methods can be used when the polypeptide, e.g., endonuclease, is relatively short (e.g., less than 10 kDa) and/or when it cannot be produced by recombinant techniques (i.e., not encoded by a nucleic acid sequence) and therefore involves different chemistry.

Solid phase synthesis procedures are well known in the art and further described by Knud J. Jensen, Pernille Tofteng Shelton and Soren L. Pedersen, Peptide Synthesis and Applications (Methods in Molecular Biology) Second Edition, Humana Press (2013); and Owen Chase, Peptides: Synthesis and Applications, Callisto Reference (2018).

In some embodiments, longer polypeptides, such as an endonuclease, may be produced by the use of recombinant methods. Methods of making a recombinant therapeutic polypeptide are routine in the art. See, in general, Voynov & Caravella (Eds.), Therapeutic Proteins: Methods and Protocols (Methods in Molecular Biology) Second Edition, Humana Press (2012); and Crommelin, Sindelar & Meibohm (Eds.), Pharmaceutical Biotechnology: Fundamentals and Applications, Springer 30 (2013).

Exemplary methods for producing a polypeptide, e.g., endonuclease, may involve expression in mammalian cells, although recombinant proteins can also be produced using insect cells, yeast, bacteria, or other cells under the control of appropriate promoters. Mammalian expression vectors may comprise nontranscribed elements such as an origin of replication, a suitable promoter, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV 40 viral genome, for example, SV 40 origin, early promoter, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

In cases where large amounts of the polypeptide, e.g., endonuclease, are desired, it can be generated using techniques such as described in Chapter 5: Large-Scale Protein Production by Gary Walsh, Proteins: Biochemistry and Biotechnology, Second Edition, John Wiley and Sons, Inc. (2015).

Various mammalian cell culture systems can be employed to express and manufacture recombinant polypeptide, e.g., endonuclease. Examples of mammalian expression systems include CHO cells, tunaCHO cells, HEK-293 cells, HCT-1080 cells, COS cells, HeLA cells and BHK cells. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). The polypeptide, e.g., endonuclease, described herein may be produced from a vector, such as a viral vector, e.g., a lentiviral vector, that encodes the recombinant endonuclease. The vector, e.g., a viral vector, comprises a nucleic acid encoding the recombinant endonuclease.

Lysis Process

In some embodiments, the step of generating the polypeptide comprises producing the polypeptide in a cell and disrupting or lysing the cell to release the produced polypeptide. In some such embodiments, the cell may be lysed by methods known in the art, such as but not limited to mechanical disruption (e.g., beads, homogenization), liquid homogenization (e.g., detergent, high salt), high frequency sound waves (e.g., sonication), freeze/thaw cycles, and manual grinding. See, for example, S. C. Bhatia, Textbook of Biotechnology, Atlantic (2006).

In some embodiments, the cell is lysed with a lysis buffer. In some such embodiments, the lysis buffer comprises a sulfate salt, for example, ammonium sulfate or sodium sulfate, or a combination thereof. In further embodiments, the lysis buffer comprises a citrate or phosphate salt, for example, ammonium citrate, ammonium phosphate, sodium citrate or sodium phosphate.

In some embodiments, the cell is physically lysed by sonication. In some embodiments, the cell is lysed by one or more methods described herein, e.g., freeze/thaw and sonication, e.g., homogenization and detergent, e.g., high salt and sonication, e.g., beads and manual grinding.

### III. Methods of Purifying the Composition

In some embodiments, the method described herein comprises purifying a composition. In some such embodiments, the method comprises a step of separating the polypeptide from at least one impurity, such as an impurity from a host cell, e.g., nucleic acids, host cell proteins, etc. In some embodiments, the polypeptide is separated from at least one impurity through pressure concentration or dialysis, and/or by contacting it with a hydrophobic material, an ion exchange material, a hydroxyapatite resin, tangential flow filtration, or any combination thereof.

### A. Pressure Concentration/Dialysis

In some embodiments, separating the polypeptide from at least one impurity comprises dialyzing the polypeptide in a selectively permeable dialysate compartment (e.g., membrane) to separate the polypeptide from at least one impurity. In some embodiments, dialysis comprises separating the endonuclease from at least one impurity by means of their unequal diffusion through a dialysate compartment, e.g., a semipermeable membrane. In some embodiments, the semipermeable membrane is a dialysis bag. In some embodiments, the semipermeable membrane is a membrane casing of defined porosity. In some embodiments, the dialysis is conducted under pressure.

A permeable dialysate compartment or membrane may be chosen that allows separation of one or more impurities to one side of the dialysate compartment and the polypeptide on an opposite side of the dialysate compartment. In some embodiments, the dialysate compartment allows one or more impurities to pass through from one side to another of the dialysate compartment (e.g., movement from inside to outside), while preventing movement of the polypeptide. Alternatively, or in addition to, the polypeptide may pass through the dialysate compartment (e.g., movement from inside to outside), while the impurities are impeded from moving through the dialysate compartment. In some embodiments, the polypeptide is dialyzed once, twice, three, four, or more times. Dialysis protocols and procedures may also include those known in the art, such as described by Baker and Low in Membrane Separation, Reference Module in Chemistry, Molecular Sciences and Chemical Engineering, Elsevier, 2015.

In some embodiments, pressure may be combined with dialysis to promote the movement of molecules across the dialysate compartment. For example, a hydrostatic pressure gradient sufficient to move at least one impurity across the dialysate compartment may be employed, while maintaining the polypeptide within the membrane. Alternatively, or in addition to, a hydrostatic pressure gradient sufficient to move the polypeptide across the membrane may be employed, while maintaining at least one impurity within the membrane. In some embodiments, a negative suction pressure is applied to the dialysate compartment.

In some embodiments, a pressure is applied to across the dialysate compartment. In some embodiments, a pressure differential from one side of the dialysate compartment to the other is about 50 mmHg to about 300 mmHg. In some embodiments, a pressure of about 50 mmHg, 55 mmHg, 60 mmHg, 65 mmHg, 70 mmHg, 75 mmHg, 80 mmHg, 85 mmHg, 90 mmHg, 95 mmHg, 100 mmHg, 105 mmHg, 110 mmHg, 115 mmHg, 120 mmHg, 125 mmHg, 130 mmHg, 135 mmHg, 140 mmHg, 145 mmHg, 150 mmHg, 155 mmHg, 160 mmHg, 165 mmHg, 170 mmHg, 175 mmHg, 180 mmHg, 185 mmHg, 190 mmHg, 195 mmHg, 200 mmHg, 205 mmHg, 210 mmHg, 215 mmHg, 220 mmHg, 225 mmHg, 230 mmHg, 235 mmHg, 240 mmHg, 245 mmHg, 250 mmHg, 255 mmHg, 260 mmHg, 265 mmHg, 270 mmHg, 275 mmHg, 280 mmHg, 285 mmHg, 290 mmHg, 295 mmHg, 300 mmHg is applied to a dialysate compartment. In some embodiments, a pressure differential of about 50 mmHg, 55 mmHg, 60 mmHg, 65 mmHg, 70 mmHg, 75 mmHg, 80 mmHg, 85 mmHg, 90 mmHg, 95 mmHg, 100 mmHg, 105 mmHg, 110 mmHg, 115 mmHg, 120 mmHg, 125 mmHg, 130 mmHg, 135 mmHg, 140 mmHg, 145 mmHg, 150 mmHg, 155 mmHg, 160 mmHg, 165 mmHg, 170 mmHg, 175 mmHg, 180 mmHg, 185 mmHg, 190 mmHg, 195 mmHg, 200 mmHg, 205 mmHg, 210 mmHg, 215 mmHg, 220 mmHg, 225 mmHg, 230 mmHg, 235 mmHg, 240 mmHg, 245 mmHg, 250 mmHg, 255 mmHg, 260 mmHg, 265 mmHg, 270 mmHg, 275 mmHg, 280 mmHg, 285 mmHg, 290 mmHg, 295 mmHg, 300 mmHg is present on at least one side of a dialysate compartment as compared to another side. In certain embodiments, the accumulative yield of the polypeptide in the concentrated fraction and/or dialyzed fraction is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the accumulative yield of the polypeptide is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%. Alternatively or in combination, a level of at least one impurity is reduced by at least about 50%, at least about 55%, at least about 60%, at least about 65%, at least about 70%, at least about 75%, at least about 80%, at least about 85%, at least about 90%, at least about 95%, or about 100% of the impurity of the starting or original composition or lysate.

In certain embodiments, a reduction of at least one impurity is at least about 0.1%, at least about 0.2%, at least about 0.5%, at least about 1.0%, at least about 2.0%, at least about 3.0%, at least about 4.0%, at least about 5.0%, at least about 10%, at least about 20%, at least about 30%, at least about 40%, at least about 50%, at least about 60%, or more. Alternatively or in combination, an accumulative reduction of the at least one impurity is at least about 0.1%, at least about 0.2%, at least about 0.5%, at least about 1.0%, at least about 2.0%, at least about 3.0%, at least about 4.0%, at least about 5.0%, at least about 10%, at least about 20%, at least about 30%, at least about 40%, at least about 50%, at least about 60%, or more of the impurity of the starting or original composition or lysate.

In certain embodiments, the percent recovery of total endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of total endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of active endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of active endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-denatured endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-denatured endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-aggregated endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-aggregated endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In some embodiments, pressure concentration comprises applying pressure to an endonuclease solution. In some embodiments, increasing pressure on the solution results in refolding of aggregates of the endonuclease.

### B. Hydrophobic Material

In some embodiments, separating the polypeptide from at least one impurity comprises contacting the polypeptide with a hydrophobic material and eluting the polypeptide with a hydrophobic material solution or a hydrophobic material solution gradient.

In one embodiment, the methods of the invention further include repeating the contacting and the eluting steps at least 1, 2, 3, 4, 5, 6, 7, 8, 9, 10, 11, 12, 13, 14, 15 or 20 times using the flow-through fraction, wash fraction, or combination thereof. In certain embodiments, the flow-through fraction and the wash fraction are combined.

In one embodiment, a portion of the polypeptide binds to the hydrophobic material. In some embodiments, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or about 100% of the polypeptides in the composition bind to the hydrophobic material. Alternatively or in combination, a substantial portion of the bound polypeptide is released from the hydrophobic material upon washing with a wash buffer. In some embodiments, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or about 100% of the amount of polypeptide bound to the hydrophobic material is released from the hydrophobic material. Alternatively or in combination, the substantial portion of the at least one impurity that binds to the hydrophobic material. In some embodiments, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the at least one impurity in the composition binds to the hydrophobic material.

In certain embodiments, the accumulative yield of the polypeptide in the flow-through fraction and/or wash fraction is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the accumulative yield of the polypeptide in any one flow-through fraction and/or wash fraction is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%. Alternatively or in combination, a level of at least one impurity of the flow-through fraction and/or wash fraction is reduced by at least about 50%, at least about 55%, at least about 60%, at least about 65%, at least about 70%, at least about 75%, at least about 80%, at least about 85%, at least about 90%, at least about 95%, or about 100% of the impurity of the starting or original composition or lysate.

In certain embodiments, a reduction of at least one impurity in any one flow-through fraction and/or wash fraction is at least about 0.1%, at least about 0.2%, at least about 0.5%, at least about 1.0%, at least about 2.0%, at least about 3.0%, at least about 4.0%, at least about 5.0%, at least about 10%, at least about 20%, at least about 30%, at least about 40%, at least about 50%, at least about 60%, or more. Alternatively or in combination, an accumulative reduction of the at least one impurity in the flow-through fraction and/or wash fraction is at least about 0.1%, at least about 0.2%, at least about 0.5%, at least about 1.0%, at least about 2.0%, at least about 3.0%, at least about 4.0%, at least about 5.0%, at least about 10%, at least about 20%, at least about 30%, at least about 40%, at least about 50%, at least about 60%, or more of the impurity of the starting or original composition or lysate.

In certain embodiments, the percent recovery of total endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of total endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of active endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of active endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-denatured endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-denatured endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-aggregated endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-aggregated endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In some embodiments, the polypeptide is contacted with a hydrophobic material. In some embodiments, the hydrophobic material comprises a hydrophobic side chain. In some embodiments, the hydrophobic side chain is an octyl, phenyl, butyl, aromatic or aliphatic side chain. In some embodiments, the aromatic side chain is a phenyl. In some embodiments, the hydrophobic material further comprises an agarose, cross-linked agarose (e.g., available under the SEPHAROSE® mark from GE Healthcare), or a cross-linked dextran gel (e.g., available under the SEPHADEX® mark from GE Healthcare). In some embodiments, the hydrophobic material comprising an aromatic side chain is phenyl high sub. In exemplary embodiments, the hydrophobic material comprises phenyl high sub, although the use of any hydrophobic material or combinations thereof demonstrated to be effective in binding the polypeptide is contemplated. The hydrophobic material used in the methods described herein may be commercially available (e.g., from GE Healthcare).

The hydrophobic material may be housed within a column, a syringe, a microfilter or a microaffinity column. The hydrophobic material may be within a chromatography column. In some embodiments, the chromatography column is an HPLC column. In some embodiments, the chromatography column is a FPLC column.

In some embodiments, the hydrophobic material is within or is a hydrophobic interaction chromatography (HIC) column. HICs are used to separate molecules based on the hydrophobicity of the molecule. Increasingly hydrophobic molecules will interact with the resin more strongly and will elute later compared to less hydrophobic molecules which will bind more loosely and elute earlier. Typically, kosmotropic salts are used to promote hydrophobic interactions of the hydrophobic molecules and the hydrophobic resin. Hydrophobic interaction resins are available in ligands of differing hydrophobicities (phenyl>octyl>butyl), varying ligand densities (high and low sub) and particle sizes. In some embodiments, the hydrophobic material comprises a butyl, pentyl, hexyl, phenyl, heptyl, or octyl resin. In some embodiments, the hydrophobic material comprises an octyl sepharose resin or capto octyl resin or octyl agarose resin. In some embodiments, the hydrophobic material comprises a heptyl polyacrylamide resin. In some embodiments, the hydrophobic material comprises phenyl sepharose resin or phenyl agarose resin or capto phenyl (high sub) resin. In some embodiments, the hydrophobic material comprises a hexyl polymethacrylate resin. In some embodiments, the hydrophobic material comprises a butyl sepharose resin.

In some embodiments, the polypeptide is eluted from the hydrophobic material with a solution or solution gradient. In some embodiments, eluting the polypeptide from the hydrophobic material with a solution or solution gradient comprises varying the conductivity of the solution or solution gradient.

In some embodiments, the solution or solution gradient comprises a salt, for example a sulfate salt, for example ammonium sulfate or sodium sulfate, or a combination thereof.

In some embodiments, the salt has a concentration of between about 50 mM and 5000 mM. In some embodiments, the salt has a concentration of between about 50 mM-1000 mM, 50 mM-2000 mM, 50 mM-3000 mM, 50 mM-4000 mM, 100 mM-5000 mM, 100 mM-4000 mM, 100 mM-3000 mM, 100 mM-2000 mM, or 100 mM-1000 mM, 1000 mM-5000 mM, 1000 mM-4000 mM, 1000 mM-3000 mM, or 1000 mM-2000 mM. In some embodiments, the salt has a concentration of about 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 975 mM, 1000 mM, 1100 mM, 1200 mM, 1300 mM, 1400 mM, 1500 mM, 1600 mM, 1700 mM, 1800 mM, 1900 mM, 2000 mM, 2100 mM, 2200 mM, 2300 mM, 2400 mM, 2500 mM, 2600 mM, 2700 mM, 2800 mM, 2900 mM, 3000 mM, 3100 mM, 3200 mM, 3300 mM, 3400 mM, 3500 mM, 3600 mM, 3700 mM, 3800 mM, 3900 mM, 4000 nM, 4100 mM, 4200 mM, 4300 mM, 4400 mM, 4500 mM, 4600 mM, 4700 mM, 4800 mM, 4900 mM, or 5000 nM.

In some embodiments, the solution or solution gradient has a pH between about 3.5 and 10.5 or between about 4.0 and 10.0, or between about 4.5 and 9.5, or between about 5.0 and 9.0, or between about 5.5 and 9.0, or between about 5.5 and 8.0, between about 5.0 and 7.0, or between about 5.5 and 7.0, or between about 5.0 and 6.0, or between about 5.5 and 6.0. In some embodiments, the solution or solution gradient has a pH of about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0.

### C. Ion Exchange Material

In some embodiments, separating the polypeptide from at least one impurity comprises contacting the polypeptide with an ion exchange material and eluting the polypeptide with an ion exchange material solution or an ion exchange material solution gradient.

In one embodiment, the methods of the invention further include repeating the contacting and the eluting steps at least 1, 2, 3, 4, 5, 6, 7, 8, 9, 10, 11, 12, 13, 14, 15 or 20 times using the flow-through fraction, wash fraction, or combination thereof. In certain embodiments, the flow-through fraction and the wash fraction are combined.

In one embodiment, a portion of the polypeptide binds to the ion exchange material. In some embodiments, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or about 100% of the polypeptides in the composition bind to the ion exchange material. Alternatively or in combination, a substantial portion of the polypeptide is released from the ion exchange material upon washing with the wash buffer. In some embodiments, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or about 100% of the amount of polypeptide bound to the ion exchange material. Alternatively or in combination, the substantial portion of the at least one impurity that binds to the ion exchange material is at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the at least one impurity in the composition binds to the ion exchange material.

In certain embodiments, the accumulative yield of the polypeptide in the flow-through fraction and/or wash fraction is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the accumulative yield of the polypeptide in any one flow-through fraction and/or wash fraction is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%. Alternatively or in combination, a level of at least one impurity of the flow-through fraction and/or wash fraction is reduced by at least about 50%, at least about 55%, at least about 60%, at least about 65%, at least about 70%, at least about 75%, at least about 80%, at least about 85%, at least about 90%, at least about 95%, or about 100% of the impurity of the starting or original composition or lysate.

In certain embodiments, a reduction of at least one impurity in any one flow-through fraction and/or wash fraction is at least about 0.1%, at least about 0.2%, at least about 0.5%, at least about 1.0%, at least about 2.0%, at least about 3.0%, at least about 4.0%, at least about 5.0%, at least about 10%, at least about 20%, at least about 30%, at least about 40%, at least about 50%, at least about 60%, or more. Alternatively or in combination, an accumulative reduction of the at least one impurity in the flow-through fraction and/or wash fraction is at least about 0.1%, at least about 0.2%, at least about 0.5%, at least about 1.0%, at least about 2.0%, at least about 3.0%, at least about 4.0%, at least about 5.0%, at least about 10%, at least about 20%, at least about 30%, at least about 40%, at least about 50%, at least about 60%, or more of the impurity of the starting or original composition or lysate.

In certain embodiments, the percent recovery of total endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of total endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of active endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of active endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-denatured endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-denatured endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-aggregated endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-aggregated endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In some embodiments, the polypeptide is contacted with an ion exchange material. In some embodiments, the ion exchange material comprises a glycosaminoglycan. In some embodiments, the ion exchange material comprising a glycosaminoglycan is anionic. In some embodiments, the ion exchange material further comprises an agarose, cross-linked agarose (e.g., available under the SEPHAROSE® mark from GE Healthcare), or a cross-linked dextran gel (e.g., available under the SEPHADEX® mark from GE Healthcare). In some embodiments, the ion exchange material is also an affinity material. In some embodiments, the glycosaminoglycan is heparin, chondroitin sulfate, dermatan sulfate, keratan sulfate, or hyaluronan. In exemplary embodiments, the ion exchange material comprises heparin SEPHAROSE®. The use of any heparin composition or combinations thereof that are demonstrated to be effective in binding the polypeptide are contemplated. The ion exchange material comprising heparin SEPHAROSE® used in the methods described herein may be commercially available, such as HITRAP® Heparin column (GE Healthcare). In exemplary embodiments, the ion exchange material comprises chondroitin sulfate, dermatan sulfate, keratan sulfate, or hyaluronan. The use of any chondroitin sulfate composition, dermatan sulfate, keratan sulfate, or hyaluronan or combinations thereof that are demonstrated to be effective in binding the polypeptide are contemplated. The chondroitin sulfate, dermatan sulfate, keratan sulfate, or hyaluronan used in the methods described herein may be commercially available.

The ion exchange material may be within a column, a syringe, a microfilter or a microaffinity column. The ion exchange material may be within a chromatography column. In some embodiments, the chromatography column is a HPLC column. In some embodiments, the chromatography column is a FPLC column. The matrix may be formed of any material suitable for the preparation of a heparin affinity matrix, and may, for example, be formulated as a resin, bead, agarose, acrylamide, glass, fiberglass, plastic, polyester, methylacrylate, cellulose, sepharose, sephacryl, and/or any other suitable material that forms a solid or semi solid support, and that permits the adsorption, ionic bonding, covalent linking, crosslinking, derivatization, or other attachment of a heparin moiety to the support matrix.

In some embodiments, the polypeptide is eluted from the ion exchange material with a solution or solution gradient. In some embodiments, eluting the polypeptide from the ion exchange material with a solution or solution gradient comprises varying the conductivity of the solution or solution gradient.

In some embodiments, the solution or solution gradient comprises a salt. In some embodiments, the salt comprises a chloride salt, for example potassium chloride, sodium chloride or ammonium chloride. In some embodiments, the salt is potassium chloride. In further embodiments, the salt comprises a sulfate salt, for example ammonium sulfate or sodium sulfate, or a combination thereof. In some embodiments, the salt has a concentration of between about 50 mM and 5000 mM. In some embodiments, the salt has a concentration of between about 50 mM-1000 mM, 50 mM-2000 mM, 50 mM-3000 mM, 50 mM-4000 mM, 100 mM-5000 mM, 100 mM-4000 mM, 100 mM-3000 mM, 100 mM-2000 mM, or 100 mM-1000 mM, 1000 mM-5000 mM, 1000 mM-4000 mM, 1000 mM-3000 mM, or 1000 mM-2000 mM. In some embodiments, the salt has a concentration of about 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 975 mM, 1000 mM, 1100 mM, 1200 mM, 1300 mM, 1400 mM, 1500 mM, 1600 mM, 1700 mM, 1800 mM, 1900 mM, 2000 mM, 2100 mM, 2200 mM, 2300 mM, 2400 mM, 2500 mM, 2600 mM, 2700 mM, 2800 mM, 2900 mM, 3000 mM, 3100 mM, 3200 mM, 3300 mM, 3400 mM, 3500 mM, 3600 mM, 3700 mM, 3800 mM, 3900 mM, 4000 nM, 4100 mM, 4200 mM, 4300 mM, 4400 mM, 4500 mM, 4600 mM, 4700 mM, 4800 mM, 4900 mM, or 5000 nM.

In some embodiments, the solution or solution gradient has a pH between about 3.5 and 10.5 or between about 4.0 and 10.0, or between about 4.5 and 9.5, or between about 5.0 and 9.0, or between about 5.5 and 9.0, or between about 5.5 and 8.0, between about 5.0 and 7.0, or between about 5.5 and 7.0, or between about 5.0 and 6.0, or between about 5.5 and 6.0. In some embodiments, the solution or solution gradient has a pH of about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0.

### D. Hydroxyapatite Resin

In some embodiments, separating the polypeptide from at least one impurity comprises contacting the polypeptide with a hydroxyapatite resin and eluting the polypeptide with a hydroxyapatite resin solution or a hydroxyapatite resin solution gradient.

In one embodiment, the methods of the invention further include repeating the contacting and the eluting steps at least 1, 2, 3, 4, 5, 6, 7, 8, 9, 10, 11, 12, 13, 14, 15 or 20 times using the flow-through fraction, wash fraction, or combination thereof. In certain embodiments, the flow-through fraction and the wash fraction are combined.

In one embodiment, a portion of the polypeptide binds to the hydroxyapatite resin. In some embodiments, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or about 100% of the polypeptides in the composition bind to the hydroxyapatite resin. Alternatively or in combination, a substantial portion of the polypeptide is released from the hydroxyapatite resin upon washing with the wash buffer. In some embodiments, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or about 100% of the amount of polypeptide bound to the hydroxyapatite resin. Alternatively or in combination, the substantial portion of the at least one impurity that binds to the ion exchange material is at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the at least one impurity in the composition binds to the hydroxyapatite resin.

In certain embodiments, the accumulative yield of the polypeptide in the flow-through fraction and/or wash fraction is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the accumulative yield of the polypeptide in any one flow-through fraction and/or wash fraction is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%. Alternatively or in combination, a level of at least one impurity of the flow-through fraction and/or wash fraction is reduced by at least about 50%, at least about 55%, at least about 60%, at least about 65%, at least about 70%, at least about 75%, at least about 80%, at least about 85%, at least about 90%, at least about 95%, or about 100% of the impurity of the starting or original composition or lysate.

In certain embodiments, a reduction of at least one impurity in any one flow-through fraction and/or wash fraction is at least about 0.1%, at least about 0.2%, at least about 0.5%, at least about 1.0%, at least about 2.0%, at least about 3.0%, at least about 4.0%, at least about 5.0%, at least about 10%, at least about 20%, at least about 30%, at least about 40%, at least about 50%, at least about 60%, or more. Alternatively or in combination, an accumulative reduction of the at least one impurity in the flow-through fraction and/or wash fraction is at least about 0.1%, at least about 0.2%, at least about 0.5%, at least about 1.0%, at least about 2.0%, at least about 3.0%, at least about 4.0%, at least about 5.0%, at least about 10%, at least about 20%, at least about 30%, at least about 40%, at least about 50%, at least about 60%, or more of the impurity of the starting or original composition or lysate.

In certain embodiments, the percent recovery of total endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of total endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of active endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of active endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-denatured endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-denatured endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-aggregated endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-aggregated endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In some embodiments, the polypeptide is contacted with a hydroxyapatite resin. In some embodiments, the hydroxyapatite resin comprises hydroxyapatite and fluorapatite, such as ceramic hydroxyapatite or ceramic fluorapatite forms in which nanocrystals are aggregated into particles and fused at high temperature to create stable ceramic microspheres suitable for chromatography applications. Commercial examples of ceramic hydroxyapatite include, but are not limited to, ceramic hydroxyapatite Type I and ceramic hydroxyapatite Type II. Unless specified, ceramic hydroxyapatite and ceramic fluorapatite refer to roughly spherical particles of any diameter, including but not limited to 10, 20, 40, and 80 micron. Hydroxyapatite Ultrogel refers to a product comprising microfragments of non-ceramic hydroxyapatite embedded in porous agarose microspheres. In some embodiments, the hydroxyapatite resin further comprises an agarose, cross-linked agarose (e.g., available under the SEPHAROSE® mark from GE Healthcare), or a cross-linked dextran gel (e.g., available under the SEPHADEX® mark from GE Healthcare). In some embodiments, the hydroxyapatite resin is also an affinity material. The choice of hydroxyapatite or fluorapatite, the type, and average particle diameter suitable for a particular application can be determined through experimentation by the skilled artisan.

The hydroxyapatite resin may be within a column, a syringe, a microfilter or a microaffinity column. The hydroxyapatite resin may be within a chromatography column. In some embodiments, the chromatography column is an HPLC column. In some embodiments, the chromatography column is a FPLC column. The matrix may be formed of any material suitable for the preparation of a heparin affinity matrix, and may, for example, be formulated as a resin, bead, agarose, acrylamide, glass, fiberglass, plastic, polyester, methylacrylate, cellulose, sepharose, sephacryl, and/or any other suitable material that forms a solid or semi solid support, and that permits the adsorption, ionic bonding, covalent linking, crosslinking, derivatization, or other attachment of a heparin moiety to the support matrix.

In some embodiments, the polypeptide is eluted from the hydroxyapatite resin with a solution or solution gradient. In some embodiments, eluting the polypeptide from the resin with a solution or solution gradient comprises varying the conductivity of the solution or solution gradient.

In some embodiments, the solution or solution gradient comprises a phosphate buffer. In some embodiments, the solution or solution gradient comprises an increasing cationic gradient, e.g., increasing N(CH₃)₄⁺, NH₄⁺, Cs⁺, Rb⁺, K⁺, Na⁺, H⁺, Ca²⁺, Mg²⁺, or Al³⁺ ions. In some embodiments, the solution or solution gradient comprises an increasing anionic gradient, e.g., increasing citrate3-, sulfate2-, phosphate2-, F⁻, Cl⁻, Br⁻, I⁻, NO₃⁻, ClO₄⁻ ions.

In some embodiments, the solution or solution gradient comprises an increasing pH.

In some embodiments, the solution or solution gradient comprises a salt. In some embodiments, the salt comprises a chloride salt, for example potassium chloride, sodium chloride or ammonium chloride. In some embodiments, the salt is potassium chloride. In further embodiments, the salt comprises a sulfate salt, for example ammonium sulfate or sodium sulfate, or a combination thereof. In some embodiments, the salt has a concentration of between about 50 mM and 5000 mM. In some embodiments, the salt has a concentration of between about 50 mM-1000 mM, 50 mM-2000 mM, 50 mM-3000 mM, 50 mM-4000 mM, 100 mM-5000 mM, 100 mM-4000 mM, 100 mM-3000 mM, 100 mM-2000 mM, or 100 mM-1000 mM, 1000 mM-5000 mM, 1000 mM-4000 mM, 1000 mM-3000 mM, or 1000 mM-2000 mM. In some embodiments, the salt has a concentration of about 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 975 mM, 1000 mM, 1100 mM, 1200 mM, 1300 mM, 1400 mM, 1500 mM, 1600 mM, 1700 mM, 1800 mM, 1900 mM, 2000 mM, 2100 mM, 2200 mM, 2300 mM, 2400 mM, 2500 mM, 2600 mM, 2700 mM, 2800 mM, 2900 mM, 3000 mM, 3100 mM, 3200 mM, 3300 mM, 3400 mM, 3500 mM, 3600 mM, 3700 mM, 3800 mM, 3900 mM, 4000 nM, 4100 mM, 4200 mM, 4300 mM, 4400 mM, 4500 mM, 4600 mM, 4700 mM, 4800 mM, 4900 mM, or 5000 nM.

In some embodiments, the solution or solution gradient has a pH between about 3.5 and 10.5 or between about 4.0 and 10.0, or between about 4.5 and 9.5, or between about 5.0 and 9.0, or between about 5.5 and 9.0, or between about 5.5 and 8.0, between about 5.0 and 7.0, or between about 5.5 and 7.0, or between about 5.0 and 6.0, or between about 5.5 and 6.0. In some embodiments, the solution or solution gradient has a pH of about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0.

### E. Tangential Flow Filtration

In some embodiments, separating the polypeptide from at least one impurity comprises the polypeptide is purified by tangential flow filtration (TFF).

TFF is different from dead-end filtration, in that the majority of the feed flow travels tangentially across the surface of the filter, rather than into the filter. The principal advantage of this is that the filter cake (which can blind the filter) is substantially washed away during the filtration process, increasing the length of time that a filter unit can be operational.

In some embodiments, the step of separating can be a continuous process, contrary to batch-wise dead-end filtration. This type of filtration is typically selected for feeds with a high proportion of small particle size solids because solid material can quickly block (blind) the filter surface with dead-end filtration. Applied pressure causes one portion of the flow stream to pass through the membrane (filtrate/permeate) while the remainder (retentate) is collected or recirculated back to a feed reservoir. The general working principle of TFF can be found in literature, see e.g. Clutterbuck et al. (Chapt. 15, Single-Pass Tangential Flow Filtration (SPTFF) in Continuous Biomanufacturing in Subramanian (Ed), Continuous Biomanufacturing (Innovative Technologies and Methods), Wiley-VCH (2017), or Rathore et. al. (Prep Biochem Biotechnol. 2011; 41(4):398-421).

In some embodiments, a pressure is applied to the flow stream. In some embodiments, a pressure differential of about 30 mmHg to about 300 mmHg is present between the flow stream and the permeate. In some embodiments, a pressure of about 30 mmHg, 35 mmHg, 40 mmHg, 45 mmHg, 50 mmHg, 55 mmHg, 60 mmHg, 65 mmHg, 70 mmHg, 75 mmHg, 80 mmHg, 85 mmHg, 90 mmHg, 95 mmHg, 100 mmHg, 105 mmHg, 110 mmHg, 115 mmHg, 120 mmHg, 125 mmHg, 130 mmHg, 135 mmHg, 140 mmHg, 145 mmHg, 150 mmHg, 155 mmHg, 160 mmHg, 165 mmHg, 170 mmHg, 175 mmHg, 180 mmHg, 185 mmHg, 190 mmHg, 195 mmHg, 200 mmHg, 205 mmHg, 210 mmHg, 215 mmHg, 220 mmHg, 225 mmHg, 230 mmHg, 235 mmHg, 240 mmHg, 245 mmHg, 250 mmHg, 255 mmHg, 260 mmHg, 265 mmHg, 270 mmHg, 275 mmHg, 280 mmHg, 285 mmHg, 290 mmHg, 295 mmHg, 300 mmHg is applied to a flow stream. In some embodiments, a pressure differential of about 50 mmHg, 55 mmHg, 60 mmHg, 65 mmHg, 70 mmHg, 75 mmHg, 80 mmHg, 85 mmHg, 90 mmHg, 95 mmHg, 100 mmHg, 105 mmHg, 110 mmHg, 115 mmHg, 120 mmHg, 125 mmHg, 130 mmHg, 135 mmHg, 140 mmHg, 145 mmHg, 150 mmHg, 155 mmHg, 160 mmHg, 165 mmHg, 170 mmHg, 175 mmHg, 180 mmHg, 185 mmHg, 190 mmHg, 195 mmHg, 200 mmHg, 205 mmHg, 210 mmHg, 215 mmHg, 220 mmHg, 225 mmHg, 230 mmHg, 235 mmHg, 240 mmHg, 245 mmHg, 250 mmHg, 255 mmHg, 260 mmHg, 265 mmHg, 270 mmHg, 275 mmHg, 280 mmHg, 285 mmHg, 290 mmHg, 295 mmHg, 300 mmHg is present between the flow stream and the permeate.

In some embodiments, the step of purifying the polypeptide with TFF also may concentrate, diafiltrate (desalting and buffer/solvent exchange), and/or fractionate the polypeptide from larger or smaller biomolecules. TFF can also be used for clarification and removal of cells or cell debris or impurities from a composition or lysate.

Membranes with different molecular weight cutoffs (MWCO) may be used for TFF. In some embodiments, ultrafiltration membranes can be used for TFF. In cartridge filters (often called hollow fiber filters), the membrane forms a set of parallel hollow fibers. The feed stream passes through the lumen of the fibers and the permeate is collected from outside the fibers. Cartridges are characterized in terms of fiber length, lumen diameter and number of fibers, as well as filter pore size. In cassette filters, several flat sheets of membrane are held apart from each other and from the cassette housing by support screens. The feed stream passes into the space between two sheets and permeate is collected from the opposite side of the sheets. Cassettes are characterized in terms of flow path length and channel height, as well as membrane pore size. The channel height is determined by the thickness of the support screen. Both cartridges and cassettes are constructed from materials chosen for mechanical strength, chemical and physical compatibility, and low levels of extractable and/or toxic compounds.

In some embodiments, the tangential flow filtration is conducted with a filter having a pore size in the range of about 3 kDa to 300 kDa, 3 kDa to 250 kDa, 3 kDa to 200 kDa, 3 kDa to 150 kDa, 3 kDa to 100 kDa, 3 kDa to 50 kDa, 3 kDa to 40 kDa, 3 kDa to 30 kDa, 50 kDa to 300 kDa, 50 kDa to 250 kDa, 50 kDa to 200 kDa, 50 kDa to 150 kDa, or 50 kDa to 100 kDa. In some embodiments, the tangential flow filtration is conducted with a filter having an average pore size of about 3 kDa, 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, or 300 kDa.

In one embodiment, a portion of the polypeptide is exposed to TFF. In some embodiments, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or about 100% of the polypeptides in the composition remain in the retentate. Alternatively or in combination, depending on the filter pore size, a substantial portion of the polypeptide permeates the filter membrane. In some embodiments, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or about 100% of the amount of polypeptide passes through the filter membrane and is in the permeate. Alternatively or in combination, the substantial portion of at least one impurity is separated from the polypeptide, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of at least one impurity in the composition is separated from the polypeptide after TFF.

In certain embodiments, the accumulative yield of the polypeptide after TFF is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, a level of at least one impurity after TFF is reduced by at least about 50%, at least about 55%, at least about 60%, at least about 65%, at least about 70%, at least about 75%, at least about 80%, at least about 85%, at least about 90%, at least about 95%, or about 100% of the impurity of the starting or original composition or lysate.

In certain embodiments, a reduction of at least one impurity after TFF is at least about 0.1%, at least about 0.2%, at least about 0.5%, at least about 1.0%, at least about 2.0%, at least about 3.0%, at least about 4.0%, at least about 5.0%, at least about 10%, at least about 20%, at least about 30%, at least about 40%, at least about 50%, at least about 60%, or more. Alternatively or in combination, an accumulative reduction of at least one impurity after TFF is at least about 0.1%, at least about 0.2%, at least about 0.5%, at least about 1.0%, at least about 2.0%, at least about 3.0%, at least about 4.0%, at least about 5.0%, at least about 10%, at least about 20%, at least about 30%, at least about 40%, at least about 50%, at least about 60%, or more of the impurity of the starting or original composition or lysate.

In certain embodiments, the percent recovery of total endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of total endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of active endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of active endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-denatured endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-denatured endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-aggregated endonuclease is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-aggregated endonuclease is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

### IV. Compositions Comprising an Endonuclease and an Endonuclease Binding Molecule

Provided is a method for generating and purifying a composition comprising a polypeptide as described herein, and further comprising contacting the polypeptide (e.g., endonuclease) with a binding moiety (e.g., endonuclease binding molecule), wherein the polypeptide and the binding moiety form a protein effector.

Also provided is a composition generated by contacting the composition comprising a polypeptide as described herein with a binding moiety, wherein the polypeptide is an endonuclease and the binding moiety is an endonuclease binding molecule to form a protein effector.

In some embodiments, the purity of the composition is at least about 50%, 55%, 60%, 65%, 70%, 75%, 80%, 85%, 90%, 91%, 92%, 93%, 94%, 95%, 96%, 97%, 98% or 99% as determined by SEC-MALS, SDS-PAGE or HPLC-RP. In further embodiments, the purity is at least about 98% of the endonuclease.

In some embodiments, the endonuclease binding molecule is a guide RNA or crRNA.

In some embodiments, the ratio of the polypeptide and the binding moiety in the protein effector is 1:1.

In certain embodiments, the percent recovery of total protein effector is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of total protein effector is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of active protein effector is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of active protein effector is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-denatured protein effector is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-denatured protein effector is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%.

In certain embodiments, the percent recovery of non-aggregated protein effector is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the polypeptide from the starting or original composition or lysate. Alternatively or in combination, the percent recovery of non-aggregated protein effector is at least about 4%, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or about 100%

In some embodiments, the A260/A280 absorbance ratio of the protein effector is from about X to about 1.5, wherein X is less than about 1.5. In some embodiments, the polypeptide, e.g., endonuclease, e.g., untagged endonuclease, e.g., Cas protein, e.g., Cas9, has an A260/A280 absorbance ratio of from about X to about 1.5, wherein X is less than about 0.8. In some embodiments, the A260/A280 absorbance ratio is less than about 2.0, 1.95, 1.9, 1.85, 1.8, 1.75, 1.7, 1.65, 1.6, 1.55, 1.5, 1.45, 1.4, 1.35, 1.3, 1.25, 1.2, 1.15, 1.1, 1.05, 1.0, 0.95, 0.9, 0.85, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, or less.

In some embodiments, the protein effector comprises Cas9 and the Cas9 monomer molecular weight is greater than 180 kDa but less than 230 kDa. In some embodiments, the Cas9 monomer molecular weight is between about 180 and 220 kDa, between about 180 and 210 kDa, between about 180 and 200 kDa, between 185 and 195 kDa or between about 180 and 190 kDa. In some embodiments, the protein effector comprises Cas9 and the Cas9 monomer molecular weight of about 180 kDa, 185 kDa, 190 kDa, 195 kDa, 200 kDa, 205 kDa, 210 kDa, 215 kDa, 220 kDa, 225 kDa, 230 kDa, or any molecular weight therebetween. In some embodiments, the Cas9 monomer molecular weight is 190 kDa. The monomer molecular weight may be measured by SEC-MALS or by other means known in the art.

In some embodiments, no greater than about 20%, 19%, 18%, 17%, 16%, 15%, 14%, 13%, 12%, 11%, 10%, 9%, 8%, 7%, 6%, 5%, 4%, 3%, 2%, 1% of the protein effector is denatured. In further embodiments, the protein effector is not denatured.

In some embodiments, the composition comprising a protein effector is about 85%, 90%, 91%, 92%, 93%, 94%, 95%, 96%, 97%, 98%, 99% or 100% free of residual unbound endonuclease binding molecule as measured by HPLC-SAX, HPLC-IP, LC-MS, LC-MS/MS or by ribogreen/oligogreen detection of permeates through molecular weight cutoff filters that would retain the protein effector but not free endonuclease binding moieties, such as unbound guide RNA.

In some embodiments, the protein effector is at a concentration of from about 2 to about 100 mg/ml, from about 5 to about 95 mg/ml, from about 6 to about 90 mg/ml, from about 7 to about 90 mg/ml, from about 8 to about 90 mg/ml, from about 9 to about 90 mg/ml from about 10 to about 90 mg/ml, from about 15 to about 85 mg/ml, from about 20 to about 80 mg/ml, from about 25 to about 75 mg/ml, from about 30 to about 70 mg/ml, from about 35 to about 65 mg/ml as measured by SOLOVPE® apparatus, solution UV detection, SEC-MALS or HPLC-RP.

In some embodiments, the composition comprising a protein effector is about 85%, 90%, 91%, 92%, 93%, 94%, 95%, 96%, 97%, 98%, 99% or 100% free of residual host contaminant proteins as measured by HPLC-RP or by quantitative ELISA/BioLayer Interferometry using immunodetection against host proteins.

In some embodiments, the composition comprising a protein effector manufactured by the methods described above comprises at least 50, 100, 150, 200, 250, 300, 350, 400, 450, 500 mg or more of the protein effector.

The methods described herein exemplify how an endonuclease or protein effector may be purified, such that the level of at least one impurity is reduced. In various embodiments, the level of at least one impurity is reduced by at least 60%, at least 70%, at least 80%, at least 90%, at least about 91%, at least about 92%, at least about 93%, at least about 94%, at least 95%, at least about 96%, at least about 97%, at least about 98%, at least about 99%, or about 100% of the impurity in the sample.

In some embodiments, the impurity is a process-related impurity or a product-related substance. In some embodiments, the process-related impurity is a host cell protein, a host cell nucleic acid, a media component, or a chromatographic material. In further embodiments, the impurity is a product-related substance, such as a charge variant, an aggregate of the polypeptide of interest, a fragment of the polypeptide of interest and a modified protein, such as a denatured protein.

In one embodiment, the at least one impurity is a host cell protein. For example, the host cell protein may be reduced by at least 0.25, at least 0.5, at least 0.75, at least 1.0, at least 1.25 or at least 1.5 log reduction fraction.

In one embodiment, the at least one impurity is a host cell nucleic acid. For example, the host cell nucleic acid may be reduced by at least 0.25, at least 0.5, at least 0.75, at least 1.0, at least 1.25 or at least 1.5 log reduction fraction.

### V. Cells

Provided is an engineered cell comprising the protein effector of any one of the preceding embodiments. The engineered cell may be embryonic stem cell, an induced pluripotent stem cell, a hematopoietic stem cell, a neuronal stem cell, and a mesenchymal stem cell, a CD4+ T cell, a lymphoid progenitor cell, myeloid progenitor cell, a macrophage, dendritic cell, gut associated lymphoid tissue cell, a hepatocyte, an islet cell, a CD34+ cell, a circulating blood cell, e.g., a reticulocyte, a myeloid progenitor cell, or a hematopoietic stem cell, a bone marrow cell (e.g., a myeloid progenitor cell, an erythroid progenitor cell, a hematopoietic stem cell, or a mesenchymal stem cell), a myeloid progenitor cell (e.g. a common myeloid progenitor (CMP) cell), erythroid progenitor cell (e.g. a megakaryocyte erythroid progenitor (MEP) cell), a hematopoietic stem cell (e.g. a long term hematopoietic stem cell (LT-HSC), a short term hematopoietic stem cell (ST-HSC), a multipotent progenitor (MPP) cell, a lineage restricted progenitor (LRP) cell), hepatocyte, an islet cell, a CD34+ cell, fibroblast, adipose cell, endothelial cell, epithelial cell, myocyte, or myoblast.

Also provided is a method for generating an engineered cell, comprising introducing the protein effector of any one of the preceding embodiments into a cell. The method of generating the engineered cell may include any of the preceding embodiments to produce the composition described herein coupled with introducing the protein effector into any of the preceding cell types to generate the engineered cell.

A variety of methods are known in the art and suitable for introduction of the protein effector into a cell. Examples of typical techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

### VI. Pharmaceutical Compositions, Formulation, Delivery and Administration

In various embodiments, the pharmaceutical compositions described herein may comprise the polypeptide, e.g., untagged endonuclease, e.g., Cas protein, e.g., Cas9, described herein. In some embodiments, the pharmaceutical compositions comprise the engineered cell described herein.

In various embodiments, the pharmaceutical compositions described herein may be formulated for delivery to a cell and/or to a subject via any route of administration.

The compositions may be administered once to the subject or, alternatively, multiple administrations may be performed over a period of time.

In some embodiments, administrations may be given as needed, e.g., for as long as symptoms associated with the disease, disorder or condition persist. In some embodiments, repeated administrations may be indicated for the remainder of the subject's life.

In various embodiments, the present disclosure includes pharmaceutical compositions described herein with a pharmaceutically acceptable excipient.

Pharmaceutically acceptable excipient includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

Pharmaceutically acceptable solid or liquid carriers may be added to enhance or stabilize the composition, or to facilitate preparation of the composition.

The pharmaceutical compositions according to the disclosure may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration.

Pharmaceutical compositions described herein may be formulated for example including a carrier, such as a pharmaceutical carrier and/or a polymeric carrier, e.g., a liposome, and delivered by known methods to a subject in need thereof (e.g., a human or non-human agricultural or domestic animal, e.g., cattle, dog, cat, horse, poultry). Such methods include transfection (e.g., lipid-mediated, cationic polymers, calcium phosphate); electroporation or other methods of membrane disruption (e.g., nucleofection) and viral delivery (e.g., lentivirus, retrovirus, adenovirus, AAV). Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi: 10.1089/hum.2015.074; and Zuris et al. Cationic lipid-mediated delivery of proteins enables efficient protein-based genome editing in vitro and in vivo. Nat Biotechnol. 2014 Oct 30;33(1):73-80.

The methods and compositions described herein may comprise a pharmaceutical composition administered by a regimen sufficient to alleviate a symptom of the disease, disorder or condition.

Provided here are pharmaceutical compositions comprising untagged endonuclease for treating a patient having a disease, a disorder or a condition, the method comprising administering to the patient an effective amount of a composition. In some embodiments, the composition further comprises a pharmaceutically acceptable carrier or adjuvant.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: Production of Cas9

This Example demonstrates production of the endonuclease, Cas9, in host cells.

To produce large quantities of Cas9, host cells are induced to produce Cas9. As shown in the following Example, Cas9 is produced in host cells.

Cells of interest (e.g., HEK-293T, CD34+, T cells, B cells, HCT-116 cells) are transfected with plasmid DNA (encoding Cas9) using a large-scale transfection procedure used in the art.

Cas9 protein expression is induced and cells are incubated at 37° C. for 16 h. Cells are harvested by centrifugation at 5000×g for 15 min at 4° C.

### Example 2: Lysis of Cas9 from Host Cells

This Example demonstrates lysis and clarification of Cas9 from host cells.

As shown in the following Example, Cas9 is harvested from producer host cells.

Four liters worth of Cas9 expressing cell pellet from Example 1 are placed on ice overnight at 4° C. All steps in the lysing process are performed on ice to keep the cells and lysates as cold as possible. Each liter worth of cell paste is resuspended in lysis buffer (25 mM HEPES, 50% saturated ammonium sulfate, 2×HALT™ Protease Inhibitor, 5 mM DTT, pH 8.0) at 5 mL of lysis buffer per gram of cell paste. The cell suspensions, 1 liter of cell paste at a time, are sonicated (Fisher Scientific, Model FB120, 120V, 50/6t0 Hz) at 85% amplitude for 30 seconds on 10 seconds off for a total of 20 minutes on. Evenly distributed in to 8 centrifugal tubes, the lysates are centrifuged (Beckman Coulter, AVANTI® JXN-10) at 100,000× g for a half hour at 4° C.

Supernatants (soluble fraction) are collected and pooled for further analysis.

### Example 3: Purification of Cas9 Through Pressure Filtration

This Example demonstrates separation of Cas9 from host cell components.

The production of large quantities of Cas9 in host cells may lead to contamination with host cell components, e.g., DNA. As shown in the following Example, Cas9 is purified from host cell components.

Purification of Cas9 from the soluble fraction of Example 2 is performed through buffer exchange using the KRi2 tangential flow filtration (TFF) system (Spectrum Laboratories) across a 50,000 NMWC hollow fiber membrane (GE Life Sciences, Fiber I.D. 0.5 mm, Surface Area: 26 cm²) at a flow rate of 35 mL/min and constant TMP of 10 psi. Initially, the Cas9 soluble fraction is diafiltrated with 10 diavolumes of storage buffer (25 mM Tris, 300 mM Sodium Chloride, 0.1 mM EDTA, 50% Glycerol (v/v), pH 7.5). The hollow fiber is washed with ((15 mg/mL*volume of buffer exchanged Cas9)/10 mg/mL) mL of storage buffer to rinse the remaining Cas9 from the TFF flow path. The residual Cas9 is combined with the buffer exchanged Cas9 to obtain purified Cas9 at a concentration of ˜10 mg/mL in the final storage buffer.

### Example 4: Purification of Cas9 with Ion Exchange Material

This Example demonstrates separation of Cas9 from host cell components.

The production of large quantities of Cas9 in host cells may lead to contamination with host cell components, e.g., DNA. As shown in the following Example, Cas9 is purified from host cell components.

Purification of Cas9 from the soluble fraction of Example 2 is performed on HiTrap Capto S (GE Life Sciences, 1 mL) and HiTrap Capto DEAE (GE Life Sciences, 1 mL) resins. The resin screens are performed using a flow velocity of 78 cm/hr.

After equilibration with 5 column volumes (CVs) of 100% cation exchange Buffer A (25 mM HEPES, pH 7.5), the HiTrap Capto S column is loaded with diluted Cas9 lysates (1:10 in cation exchange Buffer A) at a ratio of 1:1 lysates to resin. The column is washed with 5 CVs of 100% cation exchange Buffer A to remove any unbound Cas9 before a 20 CV gradient elution from 0-100% cation exchange Buffer B (25 mM HEPES, 1M NaCl, pH 7.5) is applied across the column to remove the target proteins. Following the elution, the Capto S column is washed with 5 CVs of cation exchange Buffer B to remove any residual proteins and additional column cleaning steps are performed. This method is repeated with alternative buffers to determine the binding and release profile for the HiTrap Capto DEAE, an anion exchanger resin: anion exchange Buffer A (25 mM Glycine, pH 10.5) and anion exchange Buffer B (25 mM Glycine, 1M NaCl, pH 10.5).

Target Cas9 fractions are pooled.

### Example 5: Purification of Cas9 with Ion Exchange Material

This Example demonstrates separation of Cas9 from host cell components.

The production of large quantities of Cas9 in host cells may lead to contamination with host cell components, e.g., DNA. As shown in the following Example, Cas9 is purified from host cell components.

Purification of Cas9 from the soluble fraction of Example 2 is performed with Heparin High Performance (GE Life Sciences, 26 mm×20 cm) resin. Flow velocity for the Heparin columns is 100 cm/hr.

After equilibration with 5 column volumes (CVs) of 100% Heparin Buffer A (25 mM HEPES, 100 mM Potassium Chloride, 5 mM DTT, pH 8.0), the Heparin column resin is loaded with neat clarified cell lysates from Example 2 at a ratio of 5:1 lysates to resin. After washing with 5 CVs of 100% Heparin Buffer A to remove any unbound material, the column undergoes a 10 CV 2-step elution. The elution comprises an application of 5 CVs of 45% Heparin Buffer B (25 mM HEPES, 1M Potassium Chloride, 5 mM DTT, pH 8.0), followed by 5 CVs of 75% Heparin Buffer B to the Heparin column to remove nucleic acids and target protein respectively. The Heparin column is washed with 100% Heparin Buffer B to remove any residual material along with additional column cleaning steps.

Target Cas9 fractions are pooled.

### Example 6: Purification of Cas9 with Hydrophobic Interaction Material

This Example demonstrates separation of Cas9 from host cell components.

The production of large quantities of Cas9 in host cells may lead to contamination with host cell components, e.g., DNA. As shown in the following Example, Cas9 is purified from host cell components.

In a similar manner to the ion exchange resins, purification of Cas9 from the soluble fraction of Example 2 is performed on hydrophobic interaction chromatography (HIC) resins, HiTrap Butyl Sepharose 4 Fast Flow (GE Life Sciences, 1 mL) and HiTrap Octyl Sepharose 4 Fast Flow (GE Life Sciences, 1 mL), at a flow velocity of 78 cm/hr.

After equilibration with 5 column volumes (CVs) of 100% HIC Buffer A (25 mM HEPES, 3M NaCL, pH 7.5), the Butyl/Octyl column is loaded with neat clarified cell lysates at a ratio of 1:1 lysates to resin. The column is washed with 5 CVs of 100% HIC Buffer A to remove any unbound lysates before a 5 CV one-step elution of 75% HIC Buffer B (25 mM HEPES, pH 7.5) is applied across the column to remove the target proteins. Following the elution, the Butyl/Octyl column is washed with 5 CVs of HIC Buffer B to remove any residual proteins and additional column cleaning steps are performed.

Target Cas9 fractions are pooled.

### Example 7: Purification of Cas9 with Hydrophobic Material

This Example demonstrates separation of Cas9 from host cell components.

The production of large quantities of Cas9 in host cells may lead to contamination with host cell components, e.g., DNA. As shown in the following Example, Cas9 is purified from host cell components.

Purification of Cas9 from the soluble fraction of Example 2 is performed on Phenyl High Substitution (GE Life Sciences, 16 mm×20 cm) resin. Flow velocity for the Phenyl column is 100 cm/hr.

After equilibration with 5 column volumes (CVs) of 100% HIC Buffer A (25 mM HEPES, 50% saturated ammonium sulfate, 5 mM DTT, pH 8.0), the Phenyl High Substitution column is loaded with neat clarified cell lysates from Example 2 at a ratio of 5:1 lysates to resin. The column is washed with 5 CVs of 100% HIC Buffer A to remove any unbound lysates before a 5 CV one-step elution of 75% HIC Buffer B (25 mM HEPES, 5 mM DTT, pH 8.0) is applied across the column to remove the target proteins. Following the elution, the Phenyl column is washed with 5 CVs of HIC Buffer B to remove any residual proteins and additional column cleaning steps are performed.

Target Cas9 fractions are pooled.

### Example 8: Purification of Cas9 with Hydroxyapatite

This Example demonstrates separation of Cas9 from host cell components.

The production of large quantities of Cas9 in host cells may lead to contamination with host cell components, e.g., DNA. As shown in the following Example, Cas9 is purified from host cell components.

Purification of Cas9 from the soluble fraction of Example 2 is performed on the CHT™ Ceramic Hydroxyapatite (Bio-Rad, 26 mm×20 cm) column. Flow velocity for the Hydroxyapatite column is 238 cm/hr.

After equilibration with 5 column volumes (CVs) of CHT Buffer A (100 mM Potassium Phosphate, 200 mM Potassium Chloride, pH 8.0), the CHT Hydroxyapatite column is loaded with neat clarified cell lysates from Example 2 at a ratio of 5:1 lysates to resin. A 5 CV wash using 100% CHT Buffer A is applied to the column to remove any unbound protein. Using a 10 CV 2-step elution: 5 CVs of 20% CHT Buffer B followed by 5 CVs of 60% CHT Buffer B is applied to the Hydroxyapatite column to remove undesired nucleic acids/proteins and target Cas9, respectively. A 5 CV wash of 100% CHT Buffer C as well as additional column cleaning steps are applied to the Hydroxyapatite column to remove residual material.

The target Cas9 fractions are pooled.

### Example 9: Concentration of Cas9

This Example demonstrates concentration of purified Cas9.

As shown in the following Example, Cas9 is formulated for storage.

Pooled Cas9 fractions from any one of the preceding Examples 4-8 is concentrated and buffer exchanged using the KRi2 tangential flow filtration (TFF) system (Spectrum Laboratories) across a 50,000 NMWC hollow fiber membrane (GE Life Sciences, Fiber I.D. 0.5 mm, Surface Area: 26 cm²) at a flow rate of 35 mL/min and constant TMP of 10 psi. Initially, the purified Cas9 is concentrated to roughly 15 mg/mL. Then the concentrated Cas9 is diafiltrated with 10 diavolumes of storage buffer (25 mM Tris, 300 mM Sodium Chloride, 0.1 mM EDTA, 50% Glycerol (v/v), pH 7.5). The hollow fiber is washed with ((15 mg/mL*volume of buffer exchanged Cas9)/10 mg/mL) mL of storage buffer to rinse the remaining Cas9 from the TFF flow path. The residual Cas9 is combined with the buffer exchanged Cas9 to obtain purified Cas9 at a concentration of ˜10 mg/mL in the final storage buffer.

### Example 10: Host Cell DNA Analysis of Cas9

This Example demonstrates analysis of Cas9.

Several methods exist for quantifying levels of host cell DNA after purification. Among these are double-stranded DNA analysis, hybridization techniques, qPCR, and the Threshold assay. As shown in the following Example, host cell DNA is analyzed in the composition comprising Cas9.

The double-stranded (ds) DNA quantitation assay allows measurement of the concentration of dsDNA in a sample using fluorometers or fluorescence microplate readers. An asymmetrical cyanine dye is essentially nonfluorescent, but upon binding to dsDNA, the dye exhibits>1,000-fold fluorescence enhancement. The sample may be analyzed on a fluorometer to detect a linear relationship between the fluorescence detected and the concentration of dsDNA in a sample. See also Singer V L, et al. Anal. Biochem. 249(2) 1997: 228-238.

Hybridization assays involve binding DNA probes to denatured and immobilized host cell DNA. Probes are labeled with radioactive tags or fluorescent dyes and bind to complementary targets during hybridization. Signal detection is achieved with autoradiography or by phosphor- or fluorescence-imaging systems, and the signal detected is proportional to the amount of DNA immobilized on a filter. Depending on the probe used, this assay can be either specific or nonsequence specific. See also Saunders G C, Parkes H C Analytical Molecular Biology: Quality and Validation. Royal Society of Chemistry (RSC): London, U K, 1999; ISBN 0-85404-472-8.

qPCR or real-time PCR (rtPCR) is an extension of the polymerase chain reaction (PCR) and exploits the ability to monitor the progress of PCR as it occurs (in real time) to determine the quantity of target in the reaction. Data are collected throughout the process to monitor the increase in PCR product formation, enabling quantitative determination of the starting amounts of DNA in a sample. A range of different chemistries can be used to detect host-cell DNA when using qPCR, including the commonly used SYBR® Green I dye (Molecular Probes) and sequence-specific reporters such as hybridization and 5′-nuclease (TAQMAN® assay) probes. See also Arya M, et al. Expert Rev. Mol. Diagn. 5(2) 2005: 209-219.

The Threshold total DNA assay quantitatively measures picograms of single-stranded DNA (ssDNA). This quantification is based on a capture technique whereby a biotinylated single-stranded binding (SSB) protein and an anti-ssDNA antibody conjugated to urease bind simultaneously to the single-stranded DNA present in a sample. The complexes that are formed are then captured on a biotinylated membrane in a filtration step using the strong affinity of streptavidin for biotin. The urease conjugated to the anti-ssDNA antibody is used to detect and quantify the DNA. After filtration, the membrane is placed in a reader containing the substrate urea. The urease hydrolyzes the urea, which results in a pH shift that correlates with the amount of host-cell DNA in the sample. See also Molecular Devices. The Threshold DNA Assay Kit. www.moleculardevices.com/pages/reagents/thresh_dna.html (accessed September 2007).

### Example 11: Host Cell Protein Analysis of Cas9

This Example demonstrates analysis of Cas9.

The most common method for the monitoring, detection, and measurement of host cell proteins (HCPs) during bioprocessing manufacturing and in final biotherapeutic protein formulations destined for the clinic is ELISAs and the use of orthogonal methods to ELISA for the measurement, monitoring, and identification of HCPs.

ELISAs are high throughput, sensitive, and selective assays to monitor HCP amounts in process development, manufacturing and in final product formulations. Typically, an ELISA is established using null host cell line isolates to immunize animals and generate polyclonal antibodies. The assumption is that the null cell line HCP profile will be similar to the recombinant protein producing cell lines derived from the same host and antibodies raised are likely to represent the HCP pool in the recombinant cell line fermentation harvest material. See also Tscheliessnig A L, et al. 2013 Biotechnol J 8:655-670.

A number of orthogonal analytical approaches are currently used to complement ELISA measurement and monitoring of HCPs. The simplest is probably the use of 1D- and 2D-polyacrylamide gel electrophoresis (1D/2D-PAGE), together applied to investigate HCP dynamics. See also, Hogwood C E M, et al. 2013 Biotechnol Bioeng 110:240-251. 2D-PAGE is widely used for the monitoring of HCPs during process development, particularly the approach of 2D-DIGE, whereby multiple samples can be compared on the same gel to identify those HCPs that are present or increased/decreased throughout a process. See also, Jin M, et al. 2010 Biotechnol Bioeng 105:306-316. 2D-PAGE may also be coupled with mass spectrometry analysis of excised protein spots to identify particular spots on gels. See also, Tscheliessnig A L, et al. 2013 Biotechnol J 8:655-670.

Mass spectrometry may also monitor and identify multiple protein analytes in the same sample rapidly and in a high throughput manner. Nevertheless, mass spectrometry offers the opportunity to not only monitor and measure the host cell protein and product impurity profile but the ability to identify what is, and is not, present in any particular sample. Liquid-chromatography-coupled tandem mass spectrometry (LC-MS/MS) may be applied to increase the speed of monitoring HCPs (Doneanu C E, et al. 2012 MAbs 4(1):24-44). Technologies used in wider proteomic studies, such as the labeling of peptides by methods such as iTRAQ, can enhance the coverage of HCPs detected beyond that using a standard 2D-PAGE approach.

### Example 12: Purification of Cas9

This Example demonstrates purification of Cas9 from the soluble fraction.

Purification of NLS-Cas9-NLS from the soluble fraction was performed on the AKTA Pure 25 (GE Life Sciences) over three columns: Phenyl High Substitution (GE Life Sciences, 16 mm×20 cm), Heparin High Performance (GE Life Sciences, 26 mm×20 cm) and CHT™ Hydroxyapatite (Bio-Rad, 26 mm×20 cm). Flow velocities for the Phenyl and Heparin columns was 100 cm/hr and 238 cm/hr for the Hydroxyapatite column.

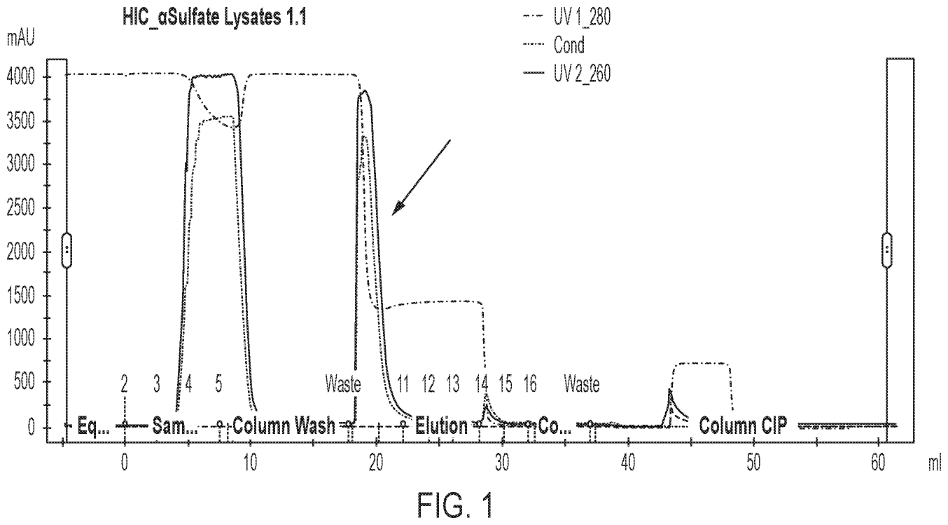

Equilibrated with 5 column volumes (CVs) of 100% HIC Buffer A (25 mM HEPES, 50% saturated ammonium sulfate, 5 mM DTT, pH 8.0), the Phenyl High Substitution column was loaded with neat clarified cell lysates at a ratio of 5:1 lysates to resin. The column was washed with 5 CVs of 100% HIC Buffer A to remove any unbound lysates before a 5 CV one-step elution of 75% HIC Buffer B (25 mM HEPES, 5 mM DTT, pH 8.0) was applied across the column to remove the target proteins. Following the elution, the Phenyl column was washed with 5 CVs of HIC Buffer B to remove any residual proteins and additional column cleaning steps were performed (FIG. 1).

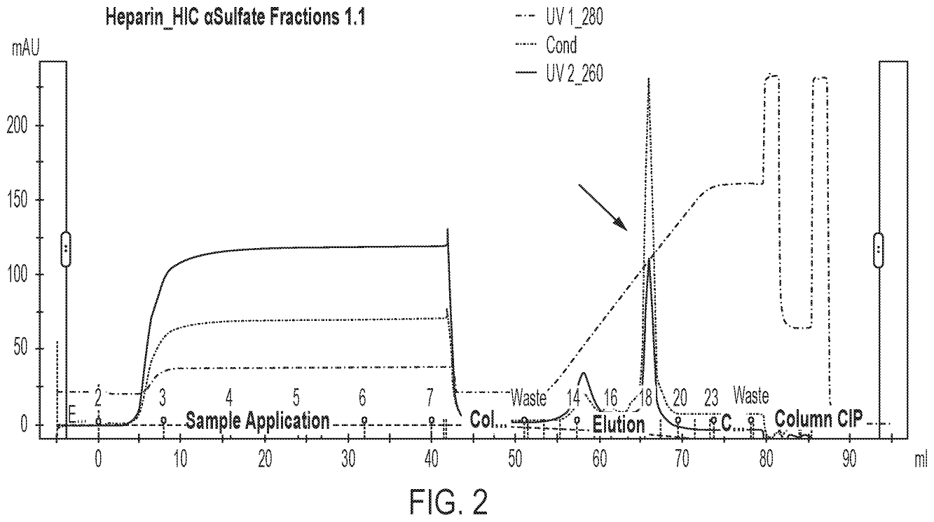

Target Phenyl High Substitution eluate was collected and diluted 1:10 with Heparin Buffer A (25 mM HEPES, 100 mM Potassium Chloride, 5 mM DTT, pH 8.0). Equilibrated with 5 column volumes (CVs) of 100% Heparin Buffer A, the Heparin column resin was loaded with the diluted HIC fractions at a ratio of 10:1 diluted HIC fractions to resin. Washed with 5 CVs of 100% Heparin Buffer A to remove any unbound material, the column underwent a 10 CV 2-step elution. The elution comprised of an application of 5 CVs of 45% Heparin Buffer B (25 mM HEPES, 1M Potassium Chloride, 5 mM DTT, pH 8.0), followed by 5 CVs of 75% Heparin Buffer B to the Heparin column to remove nucleic acids and target protein respectively. The Heparin column was washed with 100% Heparin Buffer B to remove any residual material along with additional column cleaning steps (FIG. 2).

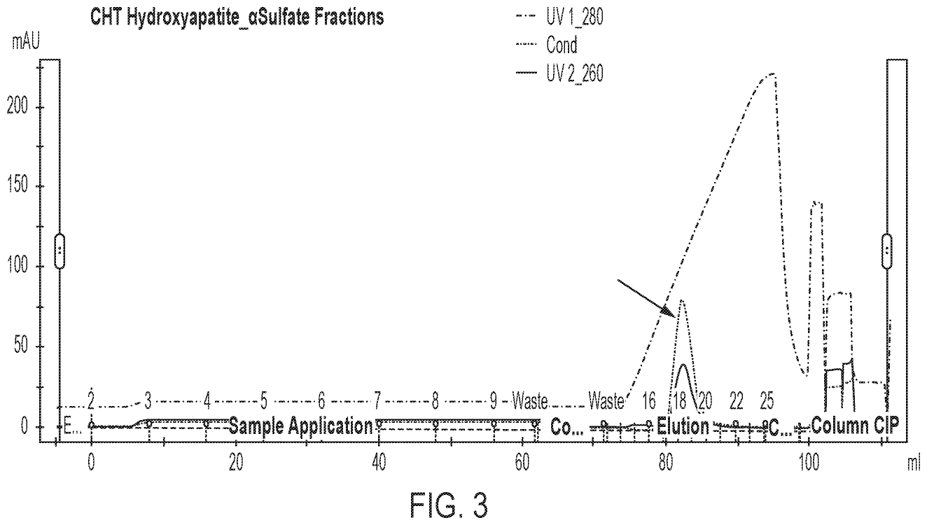

Target Heparin eluate was diluted 1:10 in CHT Buffer A (100 mM Potassium Phosphate, 200 mM Potassium Chloride, pH 8.0), then loaded, at a ratio of 14:1 (diluted Heparin fractions to resin), on to the CHT Hydroxyapatite column that was previously equilibrated with 5 CVs of CHT Buffer A. A 5 CV wash using 100% CHT Buffer A was applied to the column to remove any unbound protein. Using a 10 CV 2-step elution: 5 CVs of 20% CHT Buffer B (100 mM Potassium Phosphate, 2M Potassium Chloride, pH 8.0) followed by 5 CVs of 60% CHT Buffer B was applied to the Hydroxyapatite column to remove undesired nucleic acids/proteins and target Cas9 proteins respectively. A 5 CV wash of 100% CHT Buffer C (500 mM Potassium Phosphate, pH 7.0) as well as additional column cleaning steps were applied to the Hydroxyapatite column to remove residual material (FIG. 3).

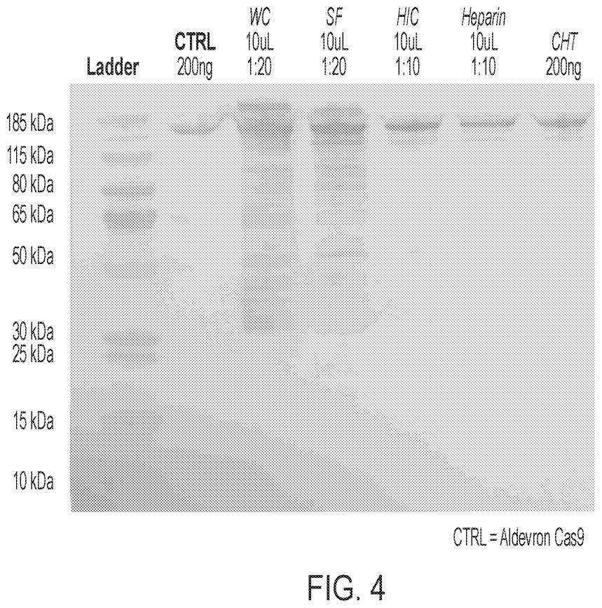

Cas9 was purified using HIC, Heparin, and CHT columns and run on an SDS-PAGE gel. Aldevron® Cas9 was used as a control for comparison. Results are depicted in FIG. 4.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising at least 100 mg of an untagged endonuclease having an A260/A280 absorbance ratio of from about X to about 0.8, wherein X is less than 0.8.

2. The composition of claim 1, wherein the endonuclease has greater than about 80% purity.

3. The composition of claim 1, wherein the endonuclease has greater than about 90% purity.

4. The composition of claim 1, wherein no greater than about 20% of the endonuclease is in the form of aggregates.

5. The composition of claim 1, wherein the composition comprises less than about 100 ng of host cell protein per mg of endonuclease.

6. The composition of claim 1, wherein the endonuclease is generated from a protein expression system.

7. The composition of claim 1, wherein the endonuclease is the polypeptide portion of a protein effector.

8. The composition of claim 1, wherein the endonuclease is Cas9 or a fusion protein thereof, or Cpf1 or a fusion protein thereof.

9. The composition of claim 8, wherein the Cas9 is a high-fidelity Cas9.

10. The composition of claim 8, wherein the Cas9 is an enzymatically inactive Cas9.

11. The composition of claim 7, wherein the polypeptide is a fusion polypeptide comprising Cas9 and another polypeptide.

12. The composition of claim 11, wherein the Cas9 is enzymatically inactive.

13. The composition of claim 11, wherein the Cas9 is enzymatically active.

14. The composition of claim 11, wherein the another polypeptide is an epigenetic-modifying agent, an exonuclease or a transcriptional modulator.

15. The composition of claim 14, wherein the epigenetic-modifying agent is a DNA methylase, a histone methyltransferase, a histone acetyltransferase, a histone deacetylase, and combinations thereof.

16. The composition of claim 8, wherein the Cas9 amino acid sequence is selected from the group consisting of SEQ ID NO: 1 to SEQ ID NO: 3.

17. The composition of claim 8, wherein the Cpf1 amino acid sequence is selected from the group consisting of SEQ ID NO: 4 to SEQ ID NO: 5.

18. The composition of claim 1, wherein the endonuclease activity of the composition when complexed with an endonuclease binding molecule at a 1:1 ratio is greater than about 20%.

19. A composition generated by contacting the composition of claim 1 with an endonuclease binding molecule, wherein the endonuclease and the endonuclease binding molecule form a protein effector.

20. The composition of claim 19, wherein the endonuclease binding molecule is a deoxyribonucleotide, a ribonucleotide, or a non-naturally occurring nucleotide.

21. The composition of claim 20, wherein the endonuclease binding molecule is a guide RNA.

22. The composition of claim 19, wherein the endonuclease activity of the composition is greater than about 20%.

23. A method for generating and purifying a composition comprising a polypeptide, the method comprising:
a) generating a composition comprising an untagged polypeptide;
b) separating the polypeptide from nucleic acids or at least one impurity in the composition by a method comprising:
i) contacting the polypeptide with a hydrophobic material comprising a hydrophobic side chain and eluting the polypeptide with a first solution or a first solution gradient; and/or
ii) contacting the polypeptide with an ion exchange material comprising a glycosaminoglycan and eluting the polypeptide with a second solution or a second solution gradient;
to obtain a composition comprising the untagged polypeptide.

24. The method of claim 23, wherein the A260/A280 absorbance ratio of the untagged polypeptide is from about X to about 0.8, wherein X is less than 0.8.

25. The method of claim 23, wherein the endonuclease activity of the composition is greater than about 20%.

26. The method of claim 23, wherein the endonuclease is an enzymatically active endonuclease or an enzymatically inactive endonuclease.

27. The method of claim 23, further comprising sonicating the composition comprising the untagged polypeptide in a lysis buffer.

28. The method of claim 27, wherein the lysis buffer comprises a sulfate salt, for example, ammonium sulfate or sodium sulfate.

29. The method of claim 27, wherein the sonication occurs after step a).

30. The method of claim 23, wherein the contacting in step i) occurs under conditions effective to permit binding of the polypeptide to the hydrophobic material comprising a hydrophobic side chain.

31. The method of claim 23, wherein the hydrophobic side chain is a octyl, phenyl, butyl, aromatic, or aliphatic side chain.

32. The method of claim 23, wherein the hydrophobic material comprising a hydrophobic side chain is phenyl high sub.

33. The method of claim 23, wherein the hydrophobic material comprising a hydrophobic side chain is comprised within a chromatography column.

34. The method of claim 23, wherein the contacting in step ii) occurs under conditions effective to permit binding of the polypeptide with the ion exchange material.

35. The method of claim 23, wherein the ion exchange material is comprised within an HPLC column.

36. The method of claim 23, wherein if step i) and step ii) are both carried out, step i) may precede step ii) or step ii) may precede step i).

37. The method of claim 23, wherein the first solution or solution gradient comprises a salt, for example a sulfate salt, for example ammonium sulfate or sodium sulfate.

38. The method of claim 23, wherein eluting the polypeptide with a first solution or solution gradient in step i) comprises varying the conductivity of the solution or solution gradient.

39. The method of claim 23, wherein eluting the polypeptide with a second solution or solution gradient in step ii) comprises varying the conductivity of the solution or solution gradient.

40. The method of claim 23, wherein the second solution or solution gradient comprises a salt, for example a sulfate salt, for example ammonium sulfate or sodium sulfate.

41. The method of claim 23, further comprising washing the untagged polypeptide on the hydrophobic material comprising a hydrophobic side chain before eluting it.

42. The method of claim 23, further comprising washing the untagged polypeptide on the ion exchange material before eluting it.

43. The method of claim 23, wherein the ion exchange material comprising a glycosaminoglycan is anionic.

44. The method of claim 23, wherein the ion exchange material comprising a glycosaminoglycan further comprises an agarose, a sepharose or a sephadex.

45. The method of claim 23, wherein the glycosaminoglycan is heparin, chondroitin sulfate, dermatan sulfate, keratan sulfate, or hyaluronan.

46. The method of claim 23, wherein the second solution or solution gradient has a pH of from about 3.5 to about 10.

47. The method of claim 23, further comprising subjecting the untagged polypeptide eluted in step i) or ii) to purification using a hydroxyapatite resin and/or tangential flow filtration.

48. The method of claim 47, wherein the tangential flow filtration is conducted with a filter having a pore size of 3 kDa to 100 kDa.

49. The method of claim 47, wherein the purification using a hydroxyapatite resin comprises the steps of:
contacting the polypeptide with a hydroxyapatite resin material and eluting the polypeptide with a third solution or a third solution gradient;
to obtain a composition comprising the untagged polypeptide.

50. The method of claim 23, wherein the polypeptide is generated from a protein expression system.

51. The method of claim 50, wherein the protein expression system comprises a cell.

52. The method of claim 50, wherein the protein expression system is cell-free.

53. The method of claim 23, wherein the untagged polypeptide is encoded by a vector that does not encode the polypeptide linked to a tag.

54. The method of claim 23, wherein the polypeptide is the polypeptide portion of a protein effector.

55. The method of claim 54, wherein the polypeptide is Cas9 or a fusion protein thereof, or Cpf1 or a fusion protein thereof.

56. The method of claim 55, wherein the Cas9 is a high-fidelity Cas9.

57. The method of claim 55, wherein the Cas9 is an enzymatically inactive Cas9.

58. The method of claim 55, wherein the polypeptide is a fusion polypeptide comprising enzymatically inactive Cas9 and another polypeptide.

59. The method of claim 58, wherein the another polypeptide is an epigenetic modifying agent or a transcriptional modulator.

60. The method of claim 59, wherein the epigenetic-modifying agent is a DNA methylase, a histone methyltransferase, a histone acetyltransferase, a histone deacetylase, and combinations thereof.

61. The method of claim 55, wherein the Cas9 amino acid sequence is selected from the group consisting of SEQ ID NO: 1 to SEQ ID NO: 3.

62. The method of claim 55, wherein the Cpf1 amino acid sequence is selected from the group consisting of SEQ ID NO: 4 to SEQ ID NO: 5.

63. The method of claim 23, further comprising contacting the polypeptide with an endonuclease binding molecule, wherein the polypeptide and the endonuclease binding molecule form a protein effector.

64. The method of claim 63, wherein the endonuclease binding molecule is a deoxyribonucleotide, a ribonucleotide, or a non-naturally occurring nucleotide.

65. The method of claim 63, wherein the endonuclease binding molecule is a guide RNA.

66. A composition comprising an untagged polypeptide that is produced by the method of any one of claims 23 to 62.

67. A composition comprising a protein effector that is produced by the method of any one of claims 63 to 65.

68. A pharmaceutical composition comprising the composition of any one of claims 1 to 22.

69. An engineered cell, comprising the protein effector of any one of claims 63 to 65.

70. The engineered cell of claim 69, wherein the engineered cell is an immune cell or precursor cell thereof, a hepatocyte, an islet cell, or a CD34+ cell.

71. A method for generating an engineered cell, comprising introducing the protein effector of any one of claims 63 to 65 into a cell.

72. The method of claim 71, wherein the engineered cell is an immune cell or precursor cell thereof, a hepatocyte, an islet cell, or a CD34+ cell.

73. The method of claim 71, wherein the protein effector is introduced into the cell by electroporation, transfection, microinjection, liposome, or a vesicle.

74. A method for treating a patient having a disease, a disorder or a condition, the method comprising administering to the patient an effective amount of a composition comprising the engineered cell of claim 69 or 70.

75. The method of claim 74, wherein the composition further comprises a pharmaceutically acceptable carrier or adjuvant.


SEQUENCE LISTING (5 sequences)

SEQ ID NO: 1 - protein, 1368 aa, Streptococcus pyogenes [sequence omitted]

SEQ ID NO: 2 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 3 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 1228 aa, Lachnospiraceae bacterium
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 5 - protein, 1307 aa, Acidaminococcus sp. (strain BV3L6)
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN